(19) United States
(12) Patent Application Publication
(10) Pub. No.: US 2014/0154342 A1
(43) Pub. Date: Jun. 5, 2014

(54) SANDALWOOD OIL AND ITS USES RELATED TO SKIN DISORDERS

(71) Applicants: Ian Clements, San Antonio, TX (US); Paul Castella, San Antonio, TX (US); Corey Levenson, San Antonio, TX (US)

(72) Inventors: Ian Clements, San Antonio, TX (US); Paul Castella, San Antonio, TX (US); Corey Levenson, San Antonio, TX (US)

(73) Assignee: SANTALIS PHARMACEUTICALS INC., San Antonio, TX (US)

(21) Appl. No.: 14/235,387

(22) PCT Filed: Jul. 27, 2012
(86) PCT No.: PCT/US2012/048599

§ 371 Date: Jan. 27, 2014

Related U.S. Application Data
(60) Provisional application No. 61/512,647, filed on Jul. 28, 2011.
(60) Provisional application No. 61/608,279, filed on Mar. 8, 2012.

(51) Int. Cl. A61K 36/185

(52) U.S. Cl. CPC A61K 36/185
USPC 424/705; 424/776; 424/779

(57) ABSTRACT

Provided herein are compositions of sandalwood heartwood oil and methods of making and using such compositions.

### SUMMARY

Provided herein are therapeutically effective compositions of sandalwood oil and kits comprising the compositions. Also provided are methods of making and using the compositions. More specifically, provided herein is a method of treating a skin disorder in a subject by administering to the subject an effective amount of a composition comprising a therapeutically effective amount of sandalwood oil, wherein the subject has a skin disorder or is at risk of developing a skin disorder.

### DESCRIPTION OF DRAWINGS

FIG. 1 shows dermal fibroblasts and epidermal keratinocytes assessed for cytotoxic response to LPS at 24 h by MTS assay. THP-1 viability after LPS treatment was assessed by trypan blue staining at 4 h. LPS treatments were from 1-6 μg/ml. Dermal fibroblasts exhibited no adverse effects from LPS treatment at doses up to 6 μg/ml. Keratinocytes exhibited decreased viability at 3 μg/ml and THP-1 cells exhibited cytotoxic effects at 2 μg/ml. All subsequent experiments were performed at 1 μg/ml.

FIG. 2 shows dermal fibroblasts and epidermal keratinocytes assessed for cytotoxic response to sandalwood oils at 24 h by MTS assay. S1 (Australian sandalwood oil) and S2 (Indian sandalwood oil) final concentrations of 5-160 μg/ml were used. Both populations exhibited acute cytotoxic effect 4 μg/ml (80 μg/μl). All subsequent experiments were performed at ≦2 μl/ml (40 μg/ml).

FIG. 3 shows THP-1 cells assessed for cytotoxic response to sandalwood oils at 4 h by trypan blue assay. S1 and S2 final concentrations of 5-160 μg/ml were used. Both oils exhibited acute cytotoxic effect at 4 μl/ml (80 μg/μl). All subsequent experiments were performed at ≦2 μl/ml (40 μg/ml).

FIG. 4 shows dermal fibroblasts assessed for cytotoxic response to Ibuprofen at 24 h by MTS assay. No cytotoxicity was evident at up to 160 μg/ml. All subsequent experiments were performed at ≦160 μg/ml.

FIG. 5 shows keratinocytes, dermal fibroblasts and THP 1 cells assessed for time-dependent expression of proinflammatory cytokines: IL-6, IL--8, MCP-1, MCP-1, ENA-78 (CXCL5) and TNFα. LPS stimulated epidermal keratinocytes expressed only IL-8. Keratinocytes were treated with LPS+S1 or S2 at 5-40 μg/ml for 24 h. Both S1 and S2 induced dose-dependent suppression of IL-8 expression in conditioned media by ELISA. IC₅₀ was achieved at the lowest tested concentration and IL-8 expression was suppressed below basal levels at 40 μg/ml.

FIG. 6 shows that LPS stimulated dermal fibroblasts expressed MCP-1, IL-6 and ENA-78. Dermal fibroblasts were treated with LPS+S1 or S2 at 5-40 μg/ml for 24 h. Both S1 and S2 induced dose-dependent suppression of MCP-1 expression in conditioned media by ELISA. IC₅₀ was less than the lowest tested concentration and IL-6 expression was suppressed to basal levels at 40 μg/ml. Ibuprofen suppressed MCP-1 expression ˜10-fold at 40-160 μg/ml.

FIG. 7 shows dermal fibroblasts treated with LPS+S1 or S2 at 5-40 μg/ml for 24 h. Both S1 and S2 induced dose-dependent suppression of IL-6 expression in conditioned media by ELISA. For S1, IC₅₀ was between 20 and 40 μg/ml while the S2 IC₅₀ was <20 μg/ml. Ibuprofen suppressed IL-6 expression with IC₅₀ of ˜40 μg/ml.

FIG. 8 shows dermal fibroblasts treated with LPS+S1 or S2 at 5-40 μg/ml for 24 h. Both S1 and S2 induced dose-dependent suppression of ENA-78 expression in conditioned media by ELISA. For S1, IC50 was between 20 and 40 μg/ml while the S2 IC₅₀ was <20 μg/ml with 40 μg/ml approximating basal expression level.

FIG. 9 shows that LPS stimulated THP-1 cells expressed MCP-1 and TNFα maximally at 4 h. THP-1 cells were treated with LPS+S1 or S2 at 5-40 μg/ml for 4 h. Both S1 and S2 induced dose-dependent suppression of MCP-1 and TNFα expression in conditioned media by ELISA. IC50 was ˜40 μg/ml for S1 and S2 on both cytokines.

FIG. 10 shows LPS stimulated dermal fibroblasts co-treated with 20 or 40 μg/ml S1 or S2 for 24 h. In addition to predicted suppression of IL-6 and MCP-1, S1 also selectively suppressed expression of GM-CSF, G-CSF, MIP-1β, RANTES and TIMP-2, but not TIMP-1 and IL-8. S2 suppressed expression of all LPS-induced cytokines on Inflammatory Array 3.

FIG. 11 shows LPS stimulated keratinocytes co-treated with 20 or 40 mg/ml S1 or S2 for 24 h. In addition to predicted suppression of IL-8, S1 also selectively suppressed LPS-induced expression of GM-CSF, G-CSF, IL-6sR, TNFR1. In addition IL-1ra, MCP-1, TIMP-1, TIMP-2, RANTES were all basally expressed and not obviously induced by LPS, but were effectively suppressed by S1 and S2.

FIG. 12 shows LPS stimulated dermal fibroblasts co-treated with 20 or 40 μg/ml S1 or S2 for 24 h. In addition to predicted suppression of ENA-78, S1 also selectively suppressed expression of all LPS-induced chemokines except Gro. MIF and MIP-3b expression were basally elevated and not substantially suppressed by S1. S2 suppressed expression of all LPS-induced and basally expressed chemokines except MIF.

FIG. 13 shows LPS stimulated keratinocytes co-treated with 20 or 40 μg/ml S1 or S2 for 24 h. LPS strongly induced expression of only ENA-78 and IP-10. Both chemokines were readily suppressed by S1 or S2. In addition, basal expression of MIF and PF4 and to a lesser extent MIP-3b expression were suppressed by S1 and S2.

FIG. 14 shows a summary of ELISA results for chemokine expression in response to LPS stimulation, treatment with S1 and treatment with S2.

FIG. 15 shows the Quantibody Human Chemokine Array 1 Map.

FIG. 16 shows the Quantibody Human Chemokine Array 3 Map.

FIG. 17 shows the Global Aesthetic Assessment Scale (GAIS) at Week 8 of acne treatment.

FIG. 18 shows absolute mean percent lesion decrease from baseline to week 8 of acne treatment.

FIG. 19 shows the median percent lesion count decrease from baseline to week 8 of acne treatment.

FIG. 20 shows the absolute change in lesion count from baseline to week 8 of acne treatment.

FIG. 21 shows the success rate of acne treatment over time as measured by GAIS.

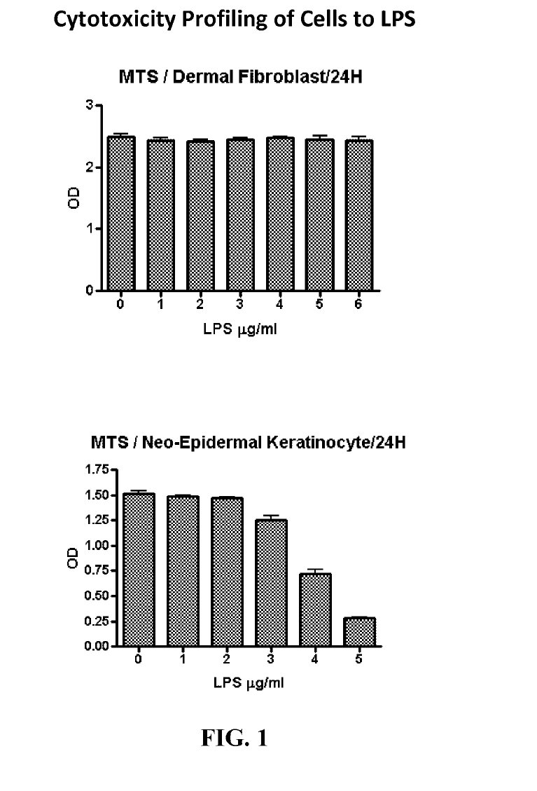

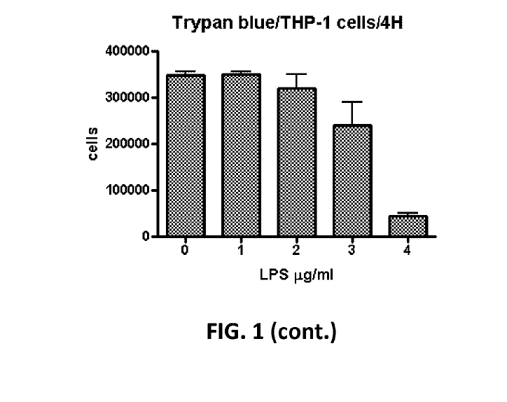

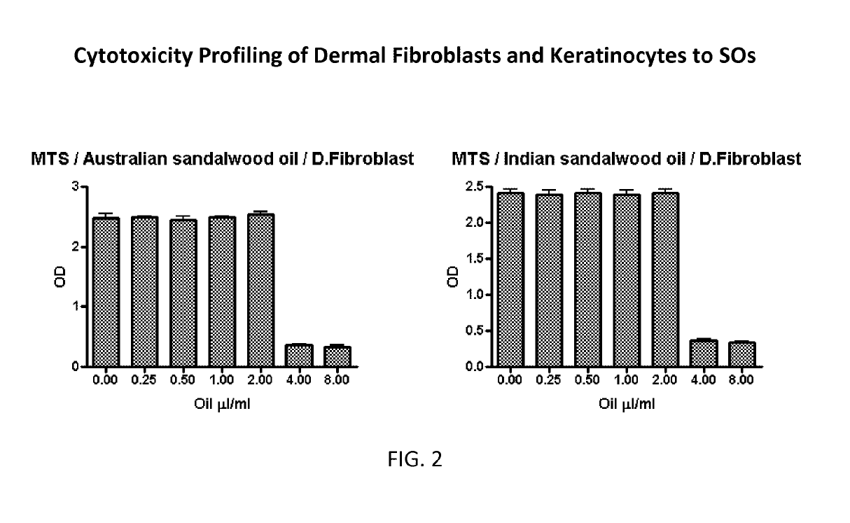

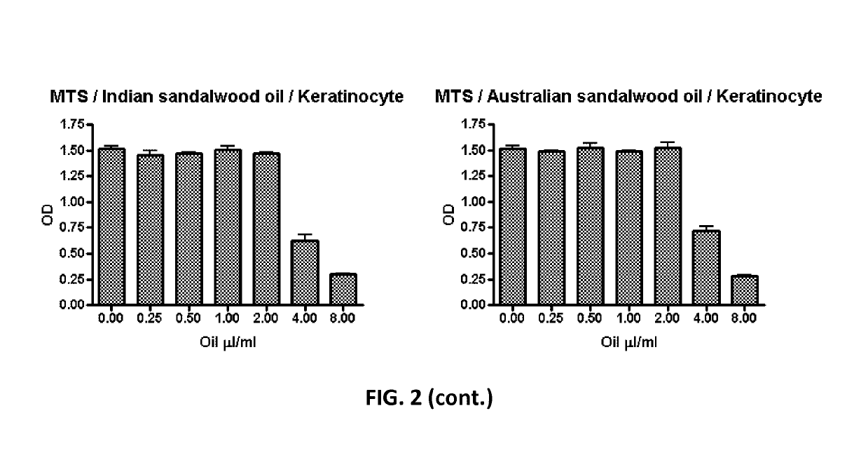

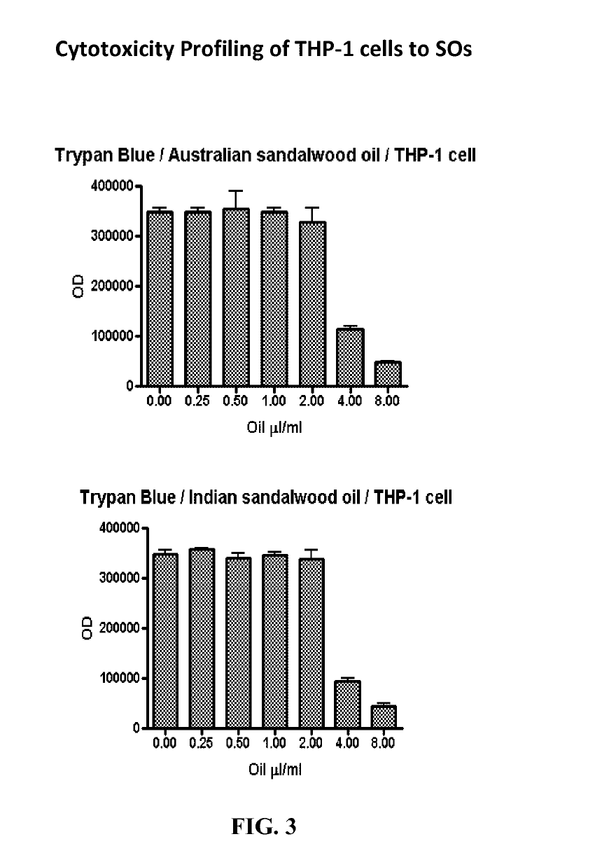

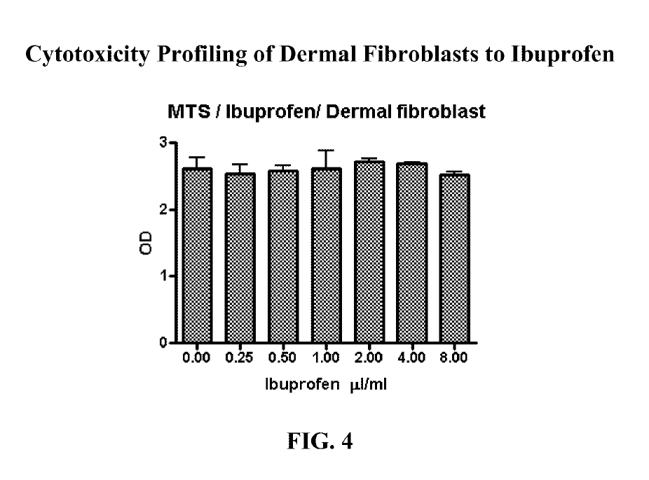

### DETAILED DESCRIPTION OF THE INVENTION

In the present compositions and methods, oil from any member of the genus Santalum can be used. For example, and not to be limiting, East Indian sandalwood (Santalum album) or West Australian sandalwood (Santalum spicatum) can be utilized in the methods and compositions set forth herein. Several other members of the genus species also have fragrant wood and are found across India, Australia, Indonesia, and the Pacific Islands. Santalum ellipticum, S. freycinetianum, and S. paniculatum, the Hawaiian sandalwoods, can also be used.

As set forth above, Santalum spicatum (West Australian sandalwood) can be used. Other species produced in Australia that can be utilized in the methods and compositions set forth herein include, but are not limited to S. acuminatum, S. lanceolatum, S. murrayanum, S. obtusifolium and S. album. The compositions set forth herein can comprise one or more sandalwood oils. The oil(s) can be from one or more members of the genus Santalum.

The components of S. spicatum and S. album species are different. A comparison of the components of steam distilled Australian and Indian sandalwood heartwood oils is presented in Table 1. The components and their percentages can vary with the extraction method.

TABLE 1
Typical Sandalwood Heartwood Oil Profiles

| Compound | S. spicatum % | S. album % |
|---|---|---|
| E nerolidol | 2.1% | 0.1% |
| Alpha-santalene | nd | 0.5% |
| Cis-alpha-(trans) bergamotene | nd | 0.7% |
| Epi-beta-santalene | nd | 1.1% |
| Beta-santalene | nd | 0.3% |
| Gamma-curcumene | nd | 0.2% |
| Dendrolasin | 1.2% | 0.2% |
| Alpha-santalol | 17.2% | 48.7% |
| Beta-bisabolol | 2.3% | 0.5% |
| Epi-alpha-bisabolol | 8% | nd |
| Z-alpha trans-bergamotol | 4.2% | 2.4% |
| Epi beta-santalol | 1.2% | 5% |
| Cis-beta-santalol | 11.4% | 20.4% |
| E,E, farnesol | 6.5% | nd |
| Cis nuciferol | 13.5% | 0.6% |
| Z-beta-curcumen-12-ol | 7.9% | 0.2% |
| cis lanceol | 2.9% | 1.5% |

The sandalwood heartwood oil can be prepared by steam distillation, supercritical CO₂ extraction, solvent extraction, hydro-distillation and combinations thereof. The sandalwood heartwood oil can also be double distilled. It is also possible to synthesize one or more of the active ingredients of sandalwood heartwood oil, as identified in Table 1 and thereafter combine individual active ingredients together.

As used herein, a sandalwood heartwood oil can be a sandalwood heartwood oil that conforms with International Organization for Standardization (ISO) specifications for the oil and therefore comprises 20-45% santalols, when derived from S. spicatum, and 57-79% santalols when derived from S. album. However, the 20-45% santalols and the 57-79% santalols are determined against the pure oil and before such oil is combined with any other solvents, excipients or active ingredients. It is understood that an efficacious preparation of sandalwood heartwood oil may have a concentration of santalols lower (or higher) than the sandalwood heartwood oil it is prepared from, and that the efficacious concentrations may be derived from sandalwood heartwood oils that are outside of the ISO specification prior to formulation. A santalol can be an α-santalol (shown below), a β-santalol (shown below), or and any other active isomers or derivatives (such as esters) thereof.

As used herein, a sandalwood heartwood oil can comprise at least about 20%, 25%, 30%, 35%, 40%, 45%, 50%, 55%, 60%, 65%, 70%, 75%, 80%, 85%, 90%, 95% , 99% santalols or any percentage in between the percentages set forth herein, when derived from S. spicatum. The sandalwood heartwood oil can comprise at least 45%, 50%, 55%, 60%, 65%, 70%, 75%, 80%, 85%, 90%, 95%, 99% santalols or any percentage in between the percentages set forth herein, when derived from S. album. As set forth above, the oil can be extracted from cultivated trees or from cell culture of tree cells.

In the methods and compositions set forth herein, the sandalwood heartwood oil can comprise the ingredients in the amounts listed in Table 1 plus or minus about 20%, and more preferably plus or minus about 10%, 5%, 2%, 1% or any percentage in between the percentages set forth herein.

It is also understood that the activity of sandalwood heartwood oil can be due to one or more components set forth in Table 1 acting either separately or together. Therefore, formulations that increase the concentration of the active component(s) and reduce the concentration of the inactive component(s) are set forth herein. Synthetic versions of the active components, or their derivatives, may be formulated in conjunction with or to replace the naturally occurring components of sandalwood heartwood oil.

In the methods and compositions set forth herein, sandalwood nut oil can be from the nut of any member of the genus Santalum. For example, and not to be limiting, East Indian sandalwood (Santalum album) or West Australian sandalwood (Santalum spicatum) nut oil can be utilized in the methods and compositions set forth herein. Nut oil from Santalum ellipticum, S. freycinetianum, and S. paniculatum, the Hawaiian sandalwoods, can also be used. Typical analytical values for sandalwood nut oil are as follows:

TABLE 2

| Fatty Acid | % concentration |
|---|---|
| Palmitic (C16.0) | 3.5% |
| Stearic (C18.0) | 1.6% |
| Oleic (C18.1 c-9) | 50.7% |
| Linoleic (C18.2 c-9 12) | 1.1% |
| Linolenic (C18.3 c-9, 12, 15) | 4.6% |
| Gondoic (C20.1 c-11) | 3% |
| Ximenynic | 35.8% |

Provided herein are therapeutically effective compositions of sandalwood oil and kits comprising the compositions. For example, provided herein is a composition comprising a therapeutically effective amount of sandalwood heartwood oil. Also provided herein is a composition comprising a therapeutically effective amount of sandalwood heartwood oil and sandalwood nut oil. As utilized herein, a therapeutically effective amount of sandalwood heartwood oil is an amount that is sufficient to reduce the effects of a skin disorder or a symptom of a skin disorder and is different from the concentration of sandalwood heartwood oil utilized to impart fragrance to a composition. The therapeutically effective amount of sandalwood heartwood oil utilized in the compositions set forth herein can be, for example, a concentration greater than about 0.3% (w/w) and up to about 10% (w/w). For example, the therapeutically effective amount can be about 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0% or any percentage (w/w) in between the percentages set forth herein.

In the compositions set forth herein, the concentration of the sandalwood nut oil can be from about 0.5% to about 10% (w/w). For example, the concentration can be about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0% or any percentage (w/w) in between the percentages set forth herein.

The compositions set forth herein can further comprise one or more over the counter active agents or ingredients approved by the Food and Drug Administration, as specified in the Code of Federal Regulations Title 21. For example, the composition can comprise any of the over-the-counter ingredients set forth under 21 C.F.R. §310.545 (see http://www.fda.gov/downloads/AboutFDA/CentersOffices/CDER/UCM135688.pdf or http://www.fda/gov/downloads/AboutFDA/CentersOffices/CDER/UCM135691.pdf incorporated herein by reference for listings of over-the-counter ingredients). The monograph specifying the concentration of each active agent for a particular indication is also provided in these listings. For example, the monograph for salicylic acid for acne treatment is listed in 21 C.F.R. §333.310(d) These include, but are not limited to, over the counter active agents for anti-acne compositions, antifungal compositions, antimicrobial compositions, as well as over-the-counter ingredients for treating dandruff, psoriasis, atopic dermatitis or seborrheic dermatitis. Over the counter active agents that can be used as an external analgesic, a skin protectant, a sunscreen or a wart remover can also be included in the compositions provided herein.

For example, and not to be limiting, the compositions set forth herein can further comprise one or more active agents selected from the group consisting of acetic acid, acetone, alcloxa, alcohol, alkyl isoquinolinium bromide, allantoin, allyl isothiocyanate, aloe vera, alum, aluminum chlorohydrex, aluminum hydroxide, aluminum sulfate, amiloxate, ammonia solution, ammoniated mercury, amyltricresois, an antibiotic, ascorbic acid, aspirin, bacitracin, basic fuchsin, beeswax, benzalkonium chloride, benzethonium chloride, benzocaine, benzoic acid, benzoxiquine, benzoyl peroxide, benzyl alcohol, bismuth subsalicylate, boric acid, calcium polysulfide, calcium thiosulfate, calcium undecylate, calcium undecylenate, calomel, camphor, camphorated metacresol, candicin, captan, chloral hydrate, chlorhydroxyquinoline (cloxyquin), chlorobutanol, chlorothymol, chloroxylenol, clioquinol, cloflucarban, clotrimazole, coal tar, colloidal oatmeal, copper undecylate, cresol, cyclomethycaine sulfate, dibenzothiophene, dichlorophen,erythromycin, estrone, ethohexadiol, eucalyptol, eugenol, fluorosalan, glycerin, haloprogin, hexachlorophene, hexylresorcinol, hydrocortisone preparations, hydrogen peroxide, iodine-containing ingredient, juniper tar, kaolin, lanolin, lauryl isoquinolinium bromide, mandelic acid, magnesium aluminum silicate, magnesium sulfate, m-cresol, methylparaben, menthol, merbromin, mercufenol chloride, mercuric chloride, mercuric oxide, mercuric salicylate, mercuric sulfide (red), mercury, mercury oleate, mercury sulfide, mercury-containing ingredient, methapyrilene hydrochloride, methyl salicylate, methylbenzethonium chloride, miconazole nitrate, mineral oil, neomycin , nitromersol, nonylphenoxypoly ethanol iodine, nystatin, oxyquinoline, oxyquinoline, oxyquinoline sulfate, PABA, para-chloromercuriphenol, peppermint oil, phenol, phenolate sodium, phenyl salicylate, phenylmercuric nitrate, pine tar, poloxamer 188, poloxamer-iodine complex, potassium, povidone-iodine, propionic acid, propylparaben, pyrilamine maleate, pyrithione zinc, resorcinol, resorcinol monoacetate, salicylamide, salicylic acid, selenium sulfide, shark liver oil, sodium borate, sodium caprylate, sodium propionate, sodium salicylate, sodium thiosulfate, sulfur, tannic acid, tetracaine, tetracaine hydrochloride, tetracycline, thimerosal, thymol, tolindate, tolnaftate, triacetin, triclocarban, triclosan, triple dye, undecoylium chlorideiodine complex, undecylenic acid, vitamin A, vitamin E, vitromersal, zinc acetate, zinc caprylate, zinc oxide, zinc propionate, zinc stearate, zinc sulfide, zinc undecylate, zyloxin, 2-ethylhexyl-4-phenylbenzopohenone-2-carboxylic acid. In addition to the active ingredients set forth herein, the compositions set forth herein can further comprise an active agent(s) set forth under 21 C.F.R. §310.545 (see http://www.fda.gov/downloads/AboutFDA/CentersOffices/CDER/UCM135688.pdf or http://www.fda.gov/downloads/AboutFDA/CentersOffices/CDER/UCM135691.pdf incorporated herein by reference for listings of over the counter ingredients)

The concentrations of the active ingredients can vary and can be any concentration within the ranges specified by the Food and Drug Administration, in the Code of Federal Regulations Title 21. For example, the concentration of an active ingredient can be from about 0.01% to about 25%. For example, the concentration can be from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.10% to about 2% (w/w), from about 0.10% to about 4% (w/w), from about 0.10% to about 6% (w/w), from about 0.1% to about 8% (w/w), from about 0.1% to about 10% (w/w), from about 0.25% to about 2% (w/w), from about 0.25% to about 4% (w/w), from about 0.25% to about 6% (w/w), from about 0.25% to about 8% (w/w), from about from about 0.25% to about 10% (w/w), from about from about 0.25% to about 12% (w/w), from about from about 0.25% to about 14% (w/w), from about from about 0.25% to about 16% (w/w), from about from about 0.25% to about 18% (w/w), from about 0.25% to about 20% (w/w), from about from about 0.25% to about 22%, from about from about 0.25% to about 24%, from about from about 0.25% to about 26%, from about from about 0.25% to about 28%, or from about from about 0.25% to about 30%. For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.0%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 29.0%, 29.1%, 29.2%, 29.3%, 29.4%, 29.5%, 29.6%, 29.7%, 29.8%, 29.9%, 30.0% or any percentage (w/w) in between the percentages set forth herein.

For example, and not to be limiting, the compositions set forth herein can comprise one or more active agents selected from the group consisting of salicylic acid, benzoyl peroxide, resorcinol, or sulfur, wherein the concentration of the salicylic acid, benzoyl peroxide, resorcinol or sulfur is from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.01% to about 11% (w/w), 0.01% to about 12% (w/w), 0.01% to about 13% (w/w), 0.01% to about 14% (w/w), 0.01% to about 15% (w/w), 0.01% to about 16% (w/w), 0.01% to about 17% (w/w), 0.01% to about 18% (w/w), 0.01% to about 19% (w/w), 0.01% to about 18% (w/w), from about 0.01% to about 20% (w/w), from about 0.10% to about 4% (w/w), from about 0.1% to about 6% (w/w), from about 0.1% to about 8% (w/w), from about 0.1% to about 10% (w/w), from about 0.1% to about 12% (w/w), from about 0.1% to about 14% (w/w), from about 0.1% to about 16% (w/w), from about 0.1% to about 18% (w/w), from about 0.1% to about 20% (w/w), from about 0.25% to about 2% (w/w), from about 0.25% to about 4% (w/w), from about 0.25% to about 6% (w/w), from about 0.25% to about 8% (w/w), from about 0.25% to about 10% (w/w), from about 0.25% to about 12% (w/w), from about 0.25% to about 14% (w/w), from about 0.25% to about 16% (w/w), from about 0.25% to about 18% (w/w), from about 0.25% to about 20% (w/w), from about from about 0.25% to about 22%, from about from about 0.25% to about 24%, from about from about 0.25% to about 26%, from about from about 0.25% to about 28%, or from about from about 0.25% to about 30%. For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0%, 20.1%, 20.2%, 20.3%, 20.4%, 20.5%, 20.6%, 20.7%, 20.8%, 20.9%, 21.0%, 21.1%, 21.2%, 21.3%, 21.4%, 21.5%, 21.6%, 21.7%, 21.8%, 21.9%, 22.0%, 22.1%, 22.2%, 22.3%, 22.4%, 22.5%, 22.6%, 22.7%, 22.8%, 22.9%, 23.0%, 23.1%, 23.2%, 23.3%, 23.4%, 23.5%, 23.6%, 23.7%, 23.8%, 23.9%, 24.0%, 24.1%, 24.2%, 24.3%, 24.4%, 24.5%, 24.6%, 24.7%, 24.8%, 24.9%, 25.0%, 25.1%, 25.2%, 25.3%, 25.4%, 25.5%, 25.6%, 25.7%, 25.8%, 25.9%, 26.0%, 26.1%, 26.2%, 26.3%, 26.4%, 26.5%, 26.6%, 26.7%, 26.8%, 26.9%, 27.0%, 27.1%, 27.2%, 27.3%, 27.4%, 27.5%, 27.6%, 27.7%, 27.8%, 27.9%, 28.0%, 28.1%, 28.2%, 28.3%, 28.4%, 28.5%, 28.6%, 28.7%, 28.8%, 28.9%, 29.0%, 29.1%, 29.2%, 29.3%, 29.4%, 29.5%, 29.6%, 29.7%, 29.8%, 29.9%, 30.0% or any percentage (w/w) in between the percentages set forth herein.

The compositions set forth herein can further comprise a fruit acid. Fruit acids that can be used include but are not limited to citric acid, glycolic acid, lactic acid, malic acid, tartaric acid and acetic acid. The fruit acid can also be a mixture of fruit acids, for example, Multifruit BSC (Arch Chemicals, Norwalk, Conn.), which is a mixture of lactic, citric, tartaric, glycolic, and malic acid extracted from plants. In certain non-limiting compositions set forth herein, the fruit acid is or includes citric acid. A fruit acid for use herein may be obtained from its natural source or may be chemically synthesized. The concentration of the fruit acid can be from about 0.01% to about 10% (w/w). For example, the concentration of the fruit acid can be from about 0.01% to about 1% (w/w), from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.10% to about 1% (w/w), from about 0.10% to about 2% (w/w), from about 0.10% to about 4% (w/w), from about 0.10% to about 6% (w/w), from about 0.1% to about 8% (w/w), from about 0.1% to about 10% (w/w), from about 0.25% to about 2% (w/w), from about 0.25% to about 4% (w/w), from about 0.25% to about 6% (w/w), from about 0.25% to about 8% (w/w) or from about from about 0.25% to about 10% (w/w). For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0% or any percentage (w/w) in between the percentages set forth herein.

The compositions set forth herein optionally also comprise a botanical extract. Botanical extracts include, plant, herbal, and fruit extracts. The botanical utilized to obtain the botanical extract may be obtained from any of the plant parts including the leaves, flowers, pulp, seeds, stems, fruit and fruit seeds, or from the whole plant. Examples of plant extracts include, but are not limited to extracts from Camellia sinensis (green tea), Melaleuca alternifolia (for example, tea tree oil), Echinacea, Centella Asiatica, onions, lemon myrtle, Irish moss, Mallow, soap bark, Yucca, sage, lavender and thyme. Curcumin compounds, and mixtures thereof can also be utilized. Examples of fruit extracts include but are not limited to grape extract, pomegranate extract, Kakadu plum (Terminalia Ferdinandiana) extract, berry extract (for example, strawberry, blueberry, blackberry, bilberry, elderberry, acai or cherry extract). Resveratrol, a polyphenolic compound from grape, berries, etc., can also be utilized. Examples of flower extracts include but are not limited to honeysuckle flower extract, ginseng flower extract, hibiscus flower extract, arnica flower extract, marigold extract, chamomile flower extract, daisy flower extract, sunflower extract rose extract, linden flower extract, elderflower extract and calendula flower extract.

Examples of herbal extracts include extracts of chamomile, rosemary, aloe, nettle, Centella asiatica, ginkgo biloba, betula, and witch hazel. The botanical extracts set forth herein can be delivered in a carrier such as water, propylene glycol, alcohol, glycerine, or butylene glycol. Additional extracts can be used, including, without limitation, white tea, grape skin, grape seed, grapefruit, grapefruit seed, grapefruit peel, citrus fruits (for example, orange, tangerine, lemon, lime, or citrus hybrids), Ginkgo biloba, soy isoflavones, soy extract, fermented soy protein, black cohosh, St. John's wort, echinacea, chamomile, rosemary, aloe extract and juice, nettle and coconut. Botanical extracts can be obtained from, for example, Active Organics (Lewisville, Tex.), New Age Botanicals (Garland, Tex.), Triarco Industries (Wayne, N.J.), and Aloecorp (Broomfield, Colo.).

In the compositions set forth herein, the concentration of a botanical extract, for example, a plant, flower or herbal extract, can be from about 0.01% to about 10% (w/w). For example, the concentration of a fruit extract, plant extract or flower extract can be from about 0.01% to about 1% (w/w), from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.10% to about 1% (w/w), from about 0.10% to about 2% (w/w), from about 0.10% to about 4% (w/w). from about 0.10% to about 6% (w/w), from about 0.10% to about 8% (w/w), from about 0.10% to about 10% (w/w), from about 0.25% to about 2% (w/w), from about 0.25% to about 4% (w/w), from about 0.25% to about 6% (w/w), from about 0.25% to about 8% (w/w) or from about from about 0.25% to about 10% (w/w). For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0% or any percentage (w/w) in between the percentages set forth herein.

Also provided herein is a composition comprising an active anti-acne agent, a fruit acid and Fusanus spicatus (Santalum spicatum) heartwood oil. Further provided is a composition comprising an active-anti-acne agent, a fruit acid, Fusanus spicatus heartwood oil and a fruit extract. Also provided is a composition comprising an active-anti-acne agent, a fruit acid, Fusanus spicatus heartwood oil, a fruit extract and a flower extract. Further provided is a composition comprising an active anti-acne agent, Fusanus spicatus heartwood oil and a flower extract. Any of the compositions set forth herein can optionally comprise lemon myrtle leaf extract. For example, provided herein is a composition comprising an active anti-acne agent, Fusanus spicatus heartwood oil, a flower extract and lemon myrtle extract.

The active anti-acne agent can be any over the counter active agents or ingredients approved by the Food and Drug Administration, as specified in the Code of Federal Regulations Title 21 monograph for acne (See, for example, 21 C.F.R. §310.545 and 21 C.F.R. §333.310 as set forth above). These include, but are not limited to, alcloxa, alkyl isoquinolinium bromide, aluminum chlorohydrex, aluminum hydroxide, benzocaine, benzoic acid, benzoyl peroxide, boric acid, calcium polysulfide, calcium thiosulfate, camphor, chlorhydroxyquinoline (cloxyquin), chloroxylenol, coal tar, dibenzothiophene, estrone, magnesium aluminum silicate, magnesium sulfate, phenol, phenolate sodium, phenyl salicylate, povidone-iodine, pyrilamine maleate, resorcinol, salicylic acid, sodium borate, sodium thiosulfate, sulfur, tetracaine hydrochloride, thymol, vitamin E, zinc oxide, zinc stearate and zinc sulfide The composition can comprise one or more active anti-acne agents. The concentrations of the active ingredients can vary and can be any concentration within the ranges specified by the Food and Drug Administration, in the Code of Federal Regulations Title 21. For example, the concentration of an active ingredient can be from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.01% to about 11% (w/w), 0.01% to about 12% (w/w), 0.01% to about 13% (w/w), 0.01% to about 14% (w/w), 0.01% to about 15% (w/w), 0.01% to about 16% (w/w), 0.01% to about 17% (w/w), 0.01% to about 18% (w/w), 0.01% to about 19% (w/w), 0.01% to about 18% (w/w), from about 0.10% to about 20% (w/w), from about 0.10% to about 4% (w/w). from about 0.10% to about 6% (w/w), from about 0.1% to about 8% (w/w), from about 0.1% to about 10% (w/w), from about 0.1% to about 12% (w/w), from about 0.1% to about 14% (w/w), from about 0.1% to about 16% (w/w), from about 0.1% to about 18% (w/w), from about 0.1% to about 20% (w/w), from about 0.25% to about 2% (w/w), from about 0.25% to about 4% (w/w), from about 0.25% to about 6% (w/w), from about 0.25% to about 8% (w/w), from about 0.25% to about 10% (w/w), from about 0.25% to about 12% (w/w), from about 0.25% to about 14% (w/w), from about 0.25% to about 16% (w/w), from about 0.25% to about 18% (w/w) or from about 0.25% to about 20% (w/w). For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, 13.0%, 13.1%, 13.2%, 13.3%, 13.4%, 13.5%, 13.6%, 13.7%, 13.8%, 13.9%, 14.0%, 14.1%, 14.2%, 14.3%, 14.4%, 14.5%, 14.6%, 14.7%, 14.8%, 14.9%, 15.0%, 15.1%, 15.2%, 15.3%, 15.4%, 15.5%, 15.6%, 15.7%, 15.8%, 15.9%, 16.0%, 16.1%, 16.2%, 16.3%, 16.4%, 16.5%, 16.6%, 16.7%, 16.8%, 16.9%, 17.0%, 17.1%, 17.2%, 17.3%, 17.4%, 17.5%, 17.6%, 17.7%, 17.8%, 17.9%, 18.0%, 18.1%, 18.2%, 18.3%, 18.4%, 18.5%, 18.6%, 18.7%, 18.8%, 18.9%, 19.0%, 19.1%, 19.2%, 19.3%, 19.4%, 19.5%, 19.6%, 19.7%, 19.8%, 19.9%, 20.0% or any percentage (w/w) in between the percentages set forth herein.

The concentration of Fusanus spicatus heartwood oil utilized in this composition can be, for example, a concentration from about 0.1% (w/w) to about 10% (w/w). For example, the therapeutically effective amount can be about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9% , 10.0% or any percentage (w/w) in between the percentages set forth herein.

Fruit acids that can be used include but are not limited to citric acid, glycolic acid, lactic acid, malic acid, tartaric acid and acetic acid. The fruit acid can also be a mixture of fruit acids, for example, Multifruit BSC (Arch Chemicals, Norwalk, Conn.), which is a mixture of lactic, citric, tartaric, glycolic, and malic acid extracted from plants. In certain non-limiting compositions set forth herein, the fruit acid is citric acid. A fruit acid for use in the invention may be obtained from its natural source or may be chemically synthesized. The concentration of the fruit acid can be from about 0.01% to about 10% (w/w). For example, the concentration of the fruit acid can be from about 0.01% to about 1% (w/w), from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.10% to about 1% (w/w), from about 0.10% to about 2% (w/w), from about 0.10% to about 4% (w/w). from about 0.10% to about 6% (w/w), from about 0.1% to about 8% (w/w), from about 0.1% to about 10% (w/w), from about 0.25% to about 2% (w/w), from about 0.25% to about 4% (w/w), from about 0.25% to about 6% (w/w), from about 0.25% to about 8% (w/w) or from about from about 0.25% to about 10% (w/w). For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0% or any percentage (w/w) in between the percentages set forth herein.

Fruit extracts and flower extracts are described above. The concentration of the fruit extract or the flower extract can be from about 0.01% to about 1% (w/w), from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.10% to about 1% (w/w), from about 0.10% to about 2% (w/w), from about 0.10% to about 4% (w/w). from about 0.10% to about 6% (w/w), from about 0.1% to about 8% (w/w), from about 0.1% to about 10% (w/w), from about 0.25% to about 2% (w/w), from about 0.25% to about 4% (w/w), from about 0.25% to about 6% (w/w), from about 0.25% to about 8% (w/w) or from about from about 0.25% to about 10% (w/w). For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0% or any percentage (w/w) in between the percentages set forth herein.

In any of the compositions comprising lemon myrtle leaf extract, the concentration of lemon myrtle leaf extract can be from about 0.01% to about 1% (w/w), from about 0.01% to about 2% (w/w), from about 0.01% to about 3% (w/w), 0.01% to about 4% (w/w), 0.01% to about 5% (w/w), 0.01% to about 6% (w/w), 0.01% to about 7% (w/w), 0.01% to about 8% (w/w), 0.01% to about 9% (w/w), 0.01% to about 10% (w/w), from about 0.10% to about 1% (w/w), from about 0.10% to about 2% (w/w), from about 0.10% to about 4% (w/w), from about 0.10% to about 6% (w/w), from about 0.1% to about 8% (w/w), from about 0.1% to about 10% (w/w), from about 0.25% to about 2% (w/w), from about 0.25% to about 4% (w/w), from about 0.25% to about 6% (w/w), from about 0.25% to about 8% (w/w) or from about from about 0.25% to about 10% (w/w). For example, the concentration can be about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0% or any percentage (w/w) in between the percentages set forth herein.

These compositions can further comprise sandalwood nut oil, for example, Fusanus spicatus nut oil. The concentration of the sandalwood nut oil can be from about 0.5% to about 10% (w/w). For example, the concentration can be about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9% , 10.0% or any percentage (w/w) in between the percentages set forth herein.

The compositions set forth herein can further comprise sandalwood powder, for example, Fusanus spicatus powder. The concentration of the sandalwood powder can be from about 0.5% to about 10% (w/w), from about 2% to about 10% (w/w) or from about 5% to about 10% (w/w). For example, the concentration can be about 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9% , 10.0% or any percentage (w/w) in between the percentages set forth herein. The compositions set forth herein can also comprise other sandalwood extracts such as hydrosols, nut proteins and other fractions.

The compositions set forth herein can include one or more solvents, including, but not limited to, a solvent(s) selected from the group consisting of water, alcohol, glycol, glycerol, glycerine, octoxyglycerin, diglycerol, butylene glycol, propylene glycol, dipropylene glycol, and vegetable oils. Examples of alcohols include but are not limited to methanol, ethanol, n-propanol, isopropyl alcohol, 2-methyl-2 propanol, hexanol, or combinations thereof. Aromatic alcohols, for example, phenoxy ethanol, benzyl alcohol, 1-phenoxy-2-propanol, and/or phenethyl alcohol, can also be used. The solvent concentration can range from about 5% to about 90% (w/w), for example, from about 10% to about 90% (w/w) or from about 20% to about 90% (w/w). For example, the concentration of the solvent can be about 5%, 10%, 15%, 20%, 25%, 30%, 35%, 40%, 45%, 50%, 55%, 60%, 65%, 70%, 75%, 80%, 85%, 90% or any percentage (w/w) in between the percentages set forth herein. When benzyl alcohol is present in a composition herein, the concentration can be from about 0.5% to about 10% (w/w), from about 0.5% to about 5% (w/w), from about 0.5% to about 4% (w/w) or from about 0.5% to about 2% (w/w).

The compositions set forth herein can also comprise pharmaceutically acceptable carriers or excipients. Other ingredients can also be included in the compositions set forth herein, which can be selected from skin cleansers, skin and hair conditioning agents, vitamins, hormones, minerals, anti-inflammatory agents, collagen and elastin synthesis boosters, UVA/UVB sunscreens, emollients, moisturizers, skin protectants, humectants, silicones, skin soothing ingredients, moisture absorbents, a powder, skin penetration enhancers, emulsifiers, solubilizers, thickeners, gelling agents, colorants, perfumes, preservatives, silica, clays, beads, luffa particles, polyethylene balls, mica, pH adjusters, processing aids, and combinations thereof. The compositions can further comprise other excipients such as hydroxypropylmethyl cellulose, cationic hydroxyethyl cellulose, ethyl cellulose, hydroxypropyl cellulose, hydroxymethyl cellulose, carboxy methyl cellulose, polyethylene oxide (polyox resins), and chitosan pyrrolidone carboxylate.

Examples of emollients include, but are not limited to, PEG 20 almond glycerides, Probutyl DB-IO, Glucam P-20, Glucam E-IO, Glucam P-10, Glucam E-20, Glucam P-20 distearate, glycerin, propylene glycol, octoxyglycerin, cetyl acetate, acetylated lanolin alcohol (e.g. , Acetulan), cetyl ether (e.g., PPG-10), myristyril ether (e.g., PPG-3), hydroxylated milk glycerides (e.g., Cremeral HMG), polyquaternium compounds, copolymers of dimethyl dialyl ammonium chloride and acrylic acid (e.g., Merquat), dipropylene glycol methyl ethers (e.g., Dowanol DPM, Dow Corning), polypropylene glycol ethers and silicon polymers. Other suitable emollients may include hydrocarbon-based emollients such as petrolatum or mineral oil, fatty ester-based emollients, such as methyl, isopropyl and butyl esters of fatty acids such as isopropyl palmitate, isopropyl myristate, isopropyl isostearate, isostearyl isostearate, diisopropyl sebacate, and propylene dipelargonate, 2-ethylhexyl isononoate, 2-ethylhexyl stearate, cetyl lactate, lauryl lactate, isopropyl lanolate, 2-ethylhexyl salicylate, cetyl myristate, oleyl myristate, oleyl stearate, oleyl oleate, hexyl laurate, and isohexyl laurate. Other moisturizers include, but are not limited to, lanolin, olive oil, cocoa butter, and shea butter.

The concentration of the excipient(s) can range from about 1.0% to about 90% (w/w), including, for example, from about 10% to about 90% (w/w) or from about 20% to about 90% (w/w). For example, the concentration of the solvent can be about 1%, 2%, 3%, 4%, 5%, 10%, 15%, 20%, 25%, 30%, 35%, 40%, 45%, 50%, 55%, 60%, 65%, 70%, 75%, 80%, 85%, 90% or any percentage (w/w) in between the percentages set forth herein. It is also understood that the combined concentration of the solvent(s) and excipient(s) can range from about 5% to about 90% (w/w), including, for example, from about 10% to about 90% (w/w) or from about 20% to about 90% (w/w). For example, the combined concentration of the solvent(s) and excipient(s) can be about 5%, 10%, 15%, 20%, 25%, 30%, 35%, 40%, 45%, 50%, 55%, 60%, 65%, 70%, 75%, 80%, 85%, 90% or any percentage (w/w) in between the percentages set forth herein.

The compositions set forth herein can be formulated as an ointment, lotion, cream, moisturizer, gel, mousse, clay mask, serum, cleanser, shaving cream, shaving gel, soap, shampoo, stick, hand sanitizer, cleansing gel, cleansing wipes, body wash, acne treatment product, diaper rash cream, conditioner, deodorant, body lotion, hand cream, topical cream, aftershave lotion, skin toner or sunscreen lotion, to name a few.

Any of the compositions set forth herein can be used to treat a skin disorder in a subject. The skin disorder can be, but is not limited to, acne, rosacea, psoriasis, eczema, dermatitis (for example, atopic dermatitis and seborrheic dermatitis), dandruff, diaper rash, a fungal infection, a viral infection, skin cancer, a precancerous skin lesion, a benign skin tumor, a mole or a skin tag. The compositions can also be used for wound healing and cleansing. Thus, any of the compositions set forth herein can be used to manufacture a medicament for the treatment of a skin disorder. For example, any of the compositions set forth herein can be used to manufacture a topical medicament for the treatment of a skin disorder selected from the group consisting of acne, rosacea, psoriasis, eczema, dermatitis (for example, atopic dermatitis and seborrheic dermatitis), dandruff, diaper rash and fungal infection. Optionally, the skin disorder excludes skin cancer.

Provided herein is a method of treating a skin disorder in a subject comprising administering any of the compositions set forth herein to the subject, where the subject has a skin disorder or is at risk of developing a skin disorder. As set forth above, the skin disorder can be, but is not limited to, acne, rosacea, psoriasis, eczema, dermatitis (for example, atopic dermatitis and seborrheic dermatitis), dandruff, diaper rash or fungal infection. Optionally, the skin disorder excludes skin cancer.

Further provided is a method of treating acne in a subject comprising administering any of the compositions set forth herein to the subject, where the subject has acne or is at risk of developing acne. As set forth above, the skin disorder can be, but is not limited to, acne, rosacea, psoriasis, eczema, dermatitis (for example, atopic dermatitis and seborrheic dermatitis), dandruff, diaper rash or fungal infection.

As used herein, the term subject can be a vertebrate, more specifically a mammal (e.g., a human, horse, pig, rabbit, dog, sheep, goat, non-human primate, cow, cat, guinea pig or rodent), a bird, a reptile or an amphibian. The term does not denote a particular age or sex. Thus, adult and newborn subjects, whether male or female, are intended to be covered. As used herein, patient or subject may be used interchangeably and can refer to a subject with a disease or disorder. The term patient or subject includes human and veterinary subjects.

As used herein the terms treatment, treat, treating or ameliorating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction or amelioration in the severity of an established disease or condition or symptom of the disease or condition. For example, and not to be limiting, a method for treating acne is considered to be a treatment if there is a 10% reduction in one or more symptoms of acne in a subject as compared to control. For example, the method for treating acne is considered to be a treatment if there is a 10% reduction in one or more symptoms of acne in a subject as compared to a control subject that did not receive a composition comprising sandalwood heartwood oil described herein. Thus, the reduction can be a 10, 20, 30, 40, 50, 60, 70, 80, 90, 100%, or any percent reduction in between 10 and 100 as compared to control levels. It is understood that treatment does not necessarily refer to a cure or complete ablation of the disease, condition, or symptoms of the disease or condition.

For the administration methods disclosed herein, each method can optionally comprise the step of diagnosing a subject with a skin disorder or at risk of developing a skin disorder. The method can also include assessing the effectiveness of the sandalwood oil composition and modifying the treatment regimen.

The sandalwood heartwood oil compositions set forth herein can be provided in a pharmaceutical composition. The compositions include a therapeutically effective amount of the sandalwood heartwood oil in combination with a pharmaceutically acceptable carrier and, in addition, may include other medicinal agents, pharmaceutical agents, carriers, or diluents.

Any appropriate route of administration may be employed, for example, administration can be systemic or local. Systemic administration includes administration via injection or infusion. Other routes of administration, such as intranasal, dermal, aerosol, vaginal, rectal or oral administration are also contemplated. Pharmaceutical compositions can be delivered locally to the area in need of treatment, for example, by topical application. By pharmaceutically acceptable carrier is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005.

Solid dosage forms for oral administration of the compositions described herein or derivatives thereof include capsules, tablets, pills, powders, and granules. In such solid dosage forms, the compositions described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alginates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compositions described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

The amount of therapeutic agent effective in treating the skin disorder can depend on the nature of the skin disorder and its associated symptoms, and can be determined by standard clinical techniques. Therefore, these amounts will vary depending on the type of skin disorder. In addition, in vitro assays can be employed to identify optimal dosage ranges. The precise dose to be employed in the formulation will also depend on the seriousness of the disease or disorder and should be decided according to the judgment of the practitioner and each subject's circumstances. The compositions describe herein can also be combined with other agents that are utilized to treat a skin disorder. For example, the compositions set forth herein can be combined with other agents utilized to treat acne (for example, adapalene, azelaic acid, benzoyl peroxide, clindamycin, erythromycin, isoretinoin, tetracycline, minocycline, doxycycline, Bactrim/Septra, oral contraceptives, sodium sulfacetamide, tazarotene, tretinoin, sprionolactone, or laser treatment), rosacea (for example, laser treatment, antibiotics or anti-hypertensives), psoriasis (for example, topical steroids, vitamin D analogues, anthralin, topical retinoids, calcinuerin inhibitors, salicylic acid, coal tar, therapeutic antibodies, or light treatment), eczema (for example, topical steroids, pimecrolimus, tacrolimus, light treatment, ciclosporin, azathioprine or methotrexate), dermatitis (for example, aclometasone, hydrocortisone, triamcinolone, clobetasol, betamethasone, mometasone or a glucocorticoid), dandruff (for example, medicated shampoo containing tar, pyrithione zinc, salicylic acid or ketoconazole), diaper rash (for example, bufexamac, eosin, topical vitamin A, talc powders or dexpanthenol ointment) or fungal infection (for example, fluconazole, voriconazole, itraconazole, ketaconazole, clotrimazole or miconazole).

Ranges may be expressed herein as from about one particular value, and/or to about another particular value. When such a range is expressed, this includes from the one particular value and/or to the other particular value. Similarly, when values are expressed as approximations, by use of the antecedent about, it will be understood that the particular value is disclosed.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

### EXAMPLES

The following are non-limiting examples of compositions that can be used to treat a skin disorder.

Formulation A

| Components | % w/w |
|---|---|
| Salicylic Acid | 0.52 |
| Citric Acid | 0.25 |
| Glyceryl Caprylate | 1.0 |
| Fusanus spicatus Heartwood Oil | 0.5 |
| Terminalia ferdinandiana Fruit Extract (and) | 0.5 |
| Glycerin (and) Alcohol Denatured | |
| Water (and) Hydrolyzed Algin (and) Zinc Sulfate | 1.0 |
| Kaolin | 52.73 |
| Montmorillonite (and) Illite (and) Kaolin | 0.5 |
| Lonicera caprifolium (Honeysuckle) Flower Extract | 1.0 |
| (And) Lonicera japonica (Honeysuckle) Flower | |
| Extract (And) Water/Aqua | |
| Fusanus spicatus Powder | 5.0 |
| Polysorbate-80 | 1.0 |
| Benzyl Alcohol | 1.0 |
| Purified Water, USP | 35.25 |

Formulation B

| Components | % w/w |
|---|---|
| Salicylic Acid | 2.05 |
| SD Alcohol 40-B | 70 |
| Fusanus spicatus Heartwood Oil | 3.0 |
| Butylene Glycol (and) Water (and) Allium cepa | 1.0 |
| (Onion) Bulb Extract | |
| Butylene Glycol (and) Water (and) Backhousia | 1.0 |
| citriodora (Lemon Myrtle) Leaf Extract | |
| Octyldodecanol, Echium Plantagineum Seed Oil, | 1.0 |
| Cardiospermum halicacabum Flower/Leaf/Vine | |
| Extract, Helianthus annuus (Sunflower) Seed Oil | |
| Unsaponfiables | |
| Hydroxypropylcellulose | 1.5 |
| Hamamelis virginiana (Witch Hazel) | 20.45 |

Formulation C

| Components | % w/w |
|---|---|
| Salicylic Acid | 0.52 |
| Butyl Avocadate | 1.0 |
| Fusanus spicatus Heartwood Oil | 2.0 |
| Terminalia ferdinandiana Fruit Extract (and) | 0.5 |
| Glycerin (and) Alcohol Denatured | |
| Water (and) Hydrolyzed Algin (and) Zinc Sulfate | 1.5 |
| Fusanus spicatus nut oil | 1.0 |
| Butylene Glycol (and) Water (and) Backhousia | 1.0 |
| citriodora (Lemon Myrtle) Leaf Extract | |
| Lonicera caprifolium (Honeysuckle) Flower Extract | 0.5 |
| (And) Lonicera japonica (Honeysuckle) Flower | |
| Extract (And) Water/Aqua | |
| Citric Acid | 0.125 |
| Polyacrylamide (and) C13-14 Isoparaffin (and) | 4.0 |
| Laureth-7 | |
| Caprylic/Capric Trigylceride | 4.0 |
| Isodecyl Neopentanoate | 5.0 |
| Purified Water, U.S. Patent and Trademark Office | 78.855 |

Formulation D

| Components | % w/w |
|---|---|
| Salicylic Acid | 0.52 |
| Fusanus spicatus Heartwood Oil | 0.25 |
| Terminalia ferdinandiana Fruit Extract (and) | 0.5 |
| Glycerin (and) Alcohol Denatured | |
| Polysorbate-80 | 5.0 |
| Butylene Glycol (and) Water (and) Backhousia | 1.0 |
| citriodora (Lemon Myrtle) Leaf Extract | |
| Lonicera caprifolium (Honeysuckle) Flower Extract | 0.5 |
| (And) Lonicera japonica (Honeysuckle) Flower | |
| Extract (And) Water/Aqua | |
| Citric Acid | 0.125 |
| Lauramidopropyl Betaine | 20 |
| Sodium Laurylglucosides Hydroxypropylsulfonate | 8.0 |
| Tangerine Grapefruit Orange | 0.4 |
| Purified Water, U.S. Patent and Trademark Office | 63.705 |

### Anti-Inflammatory Effects of Sandalwood Oil

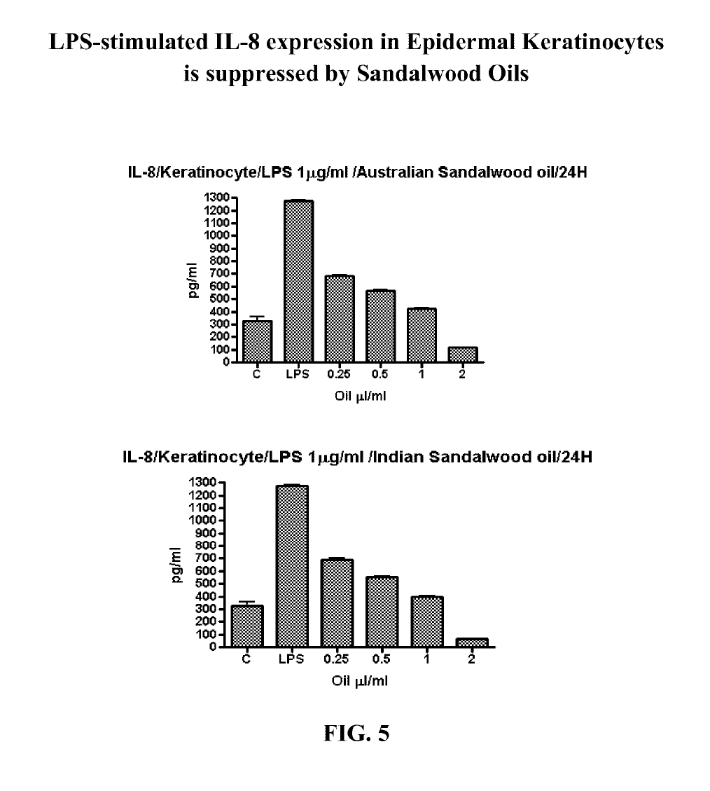

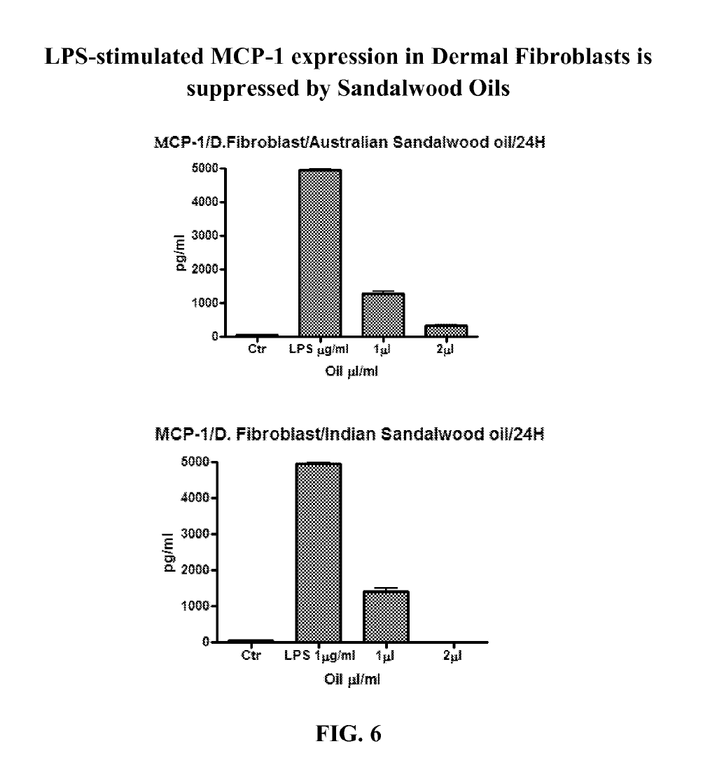

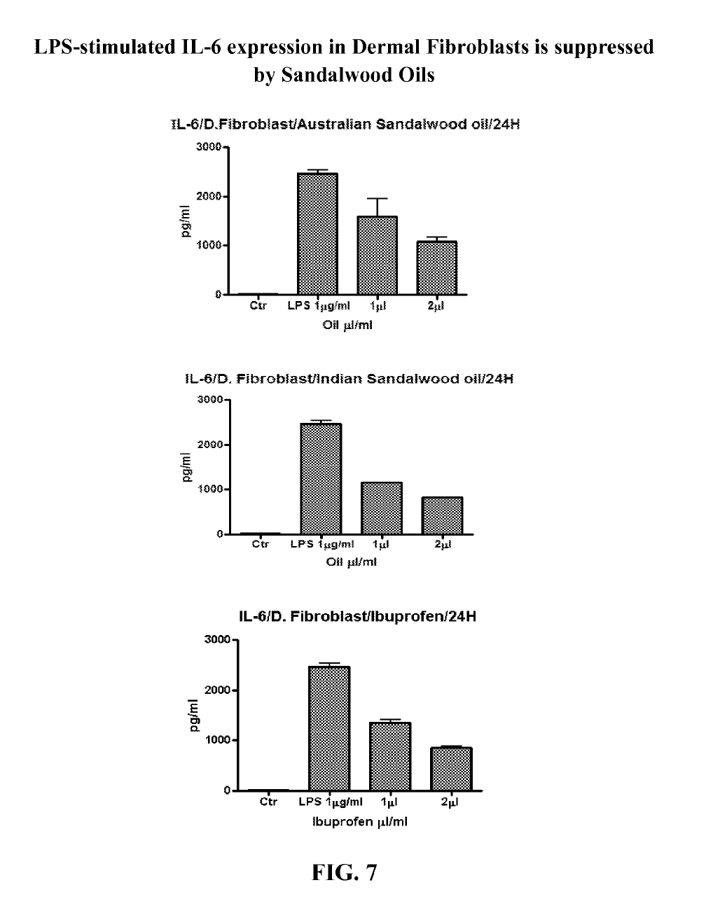

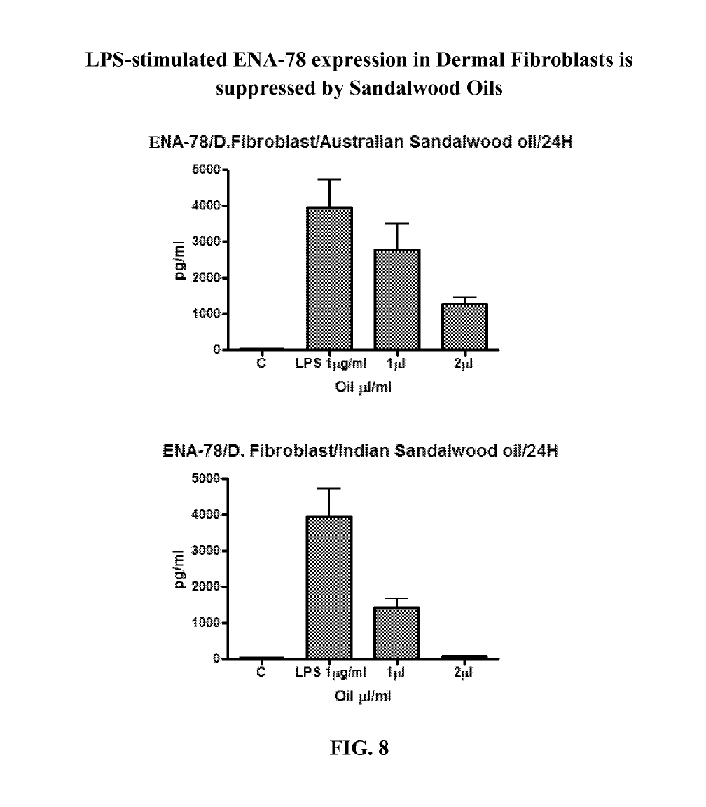

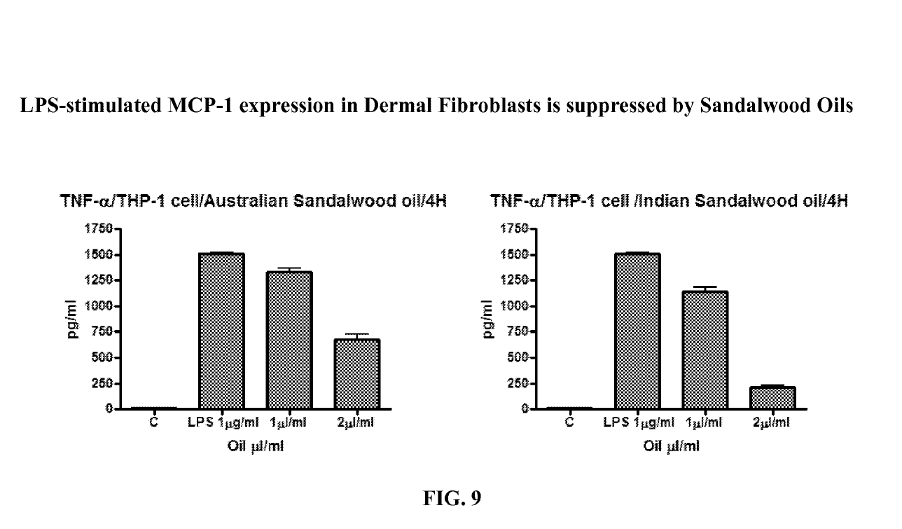

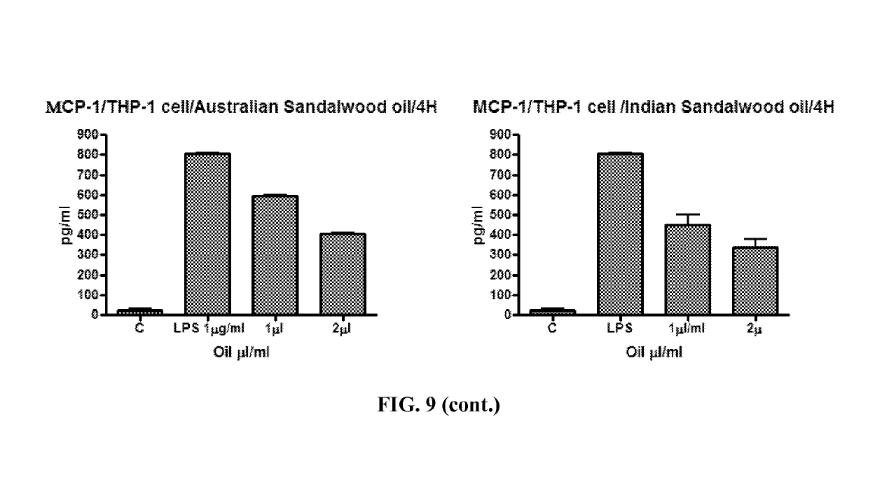

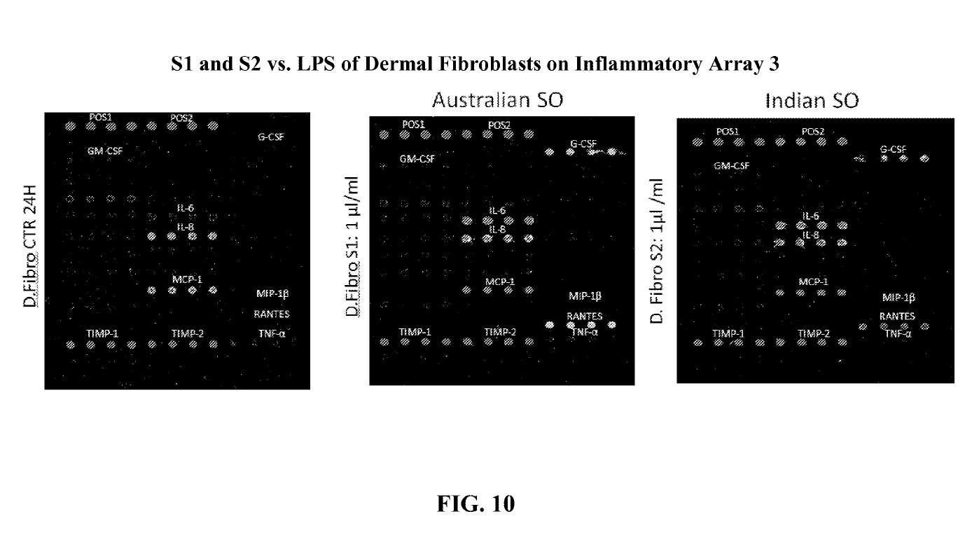

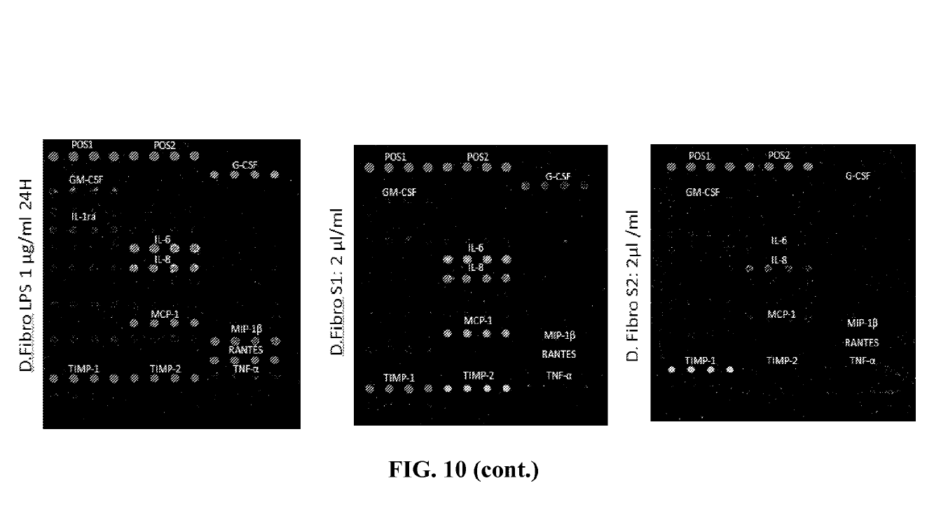

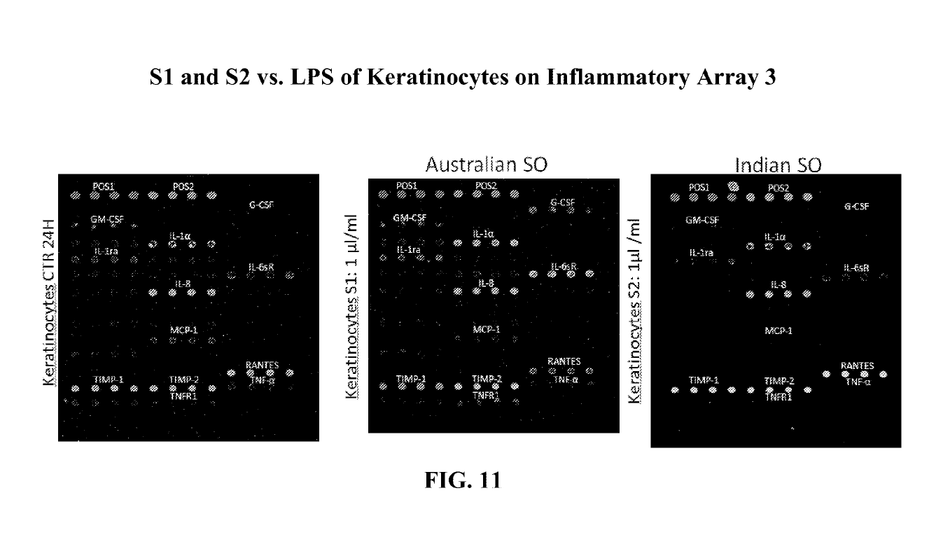

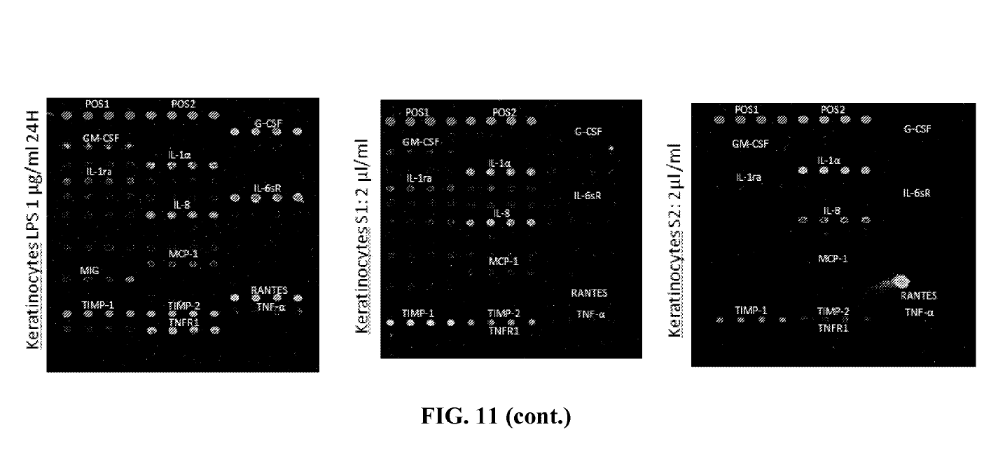

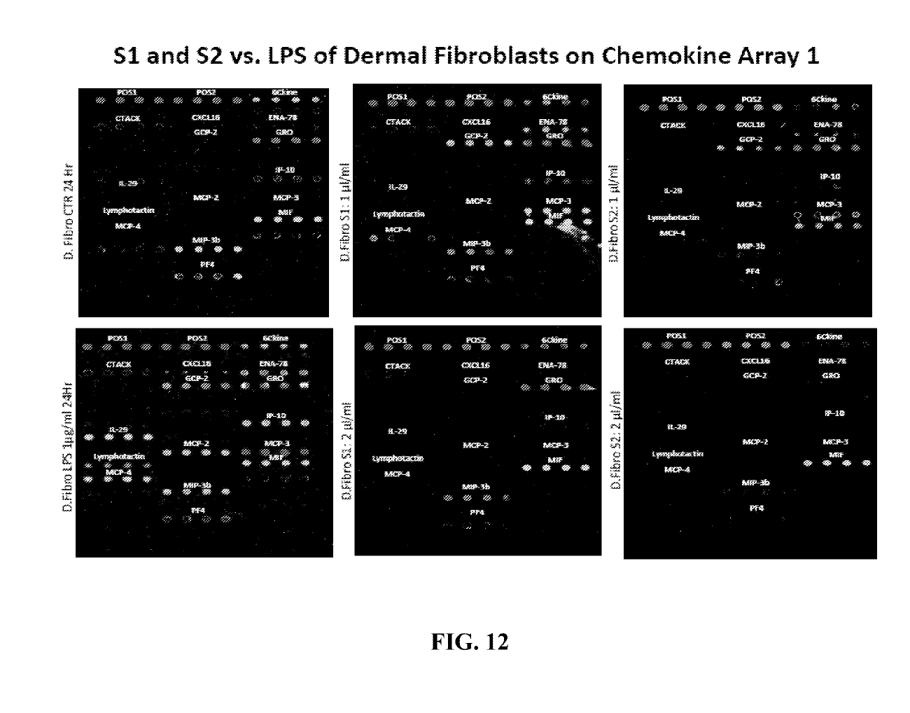

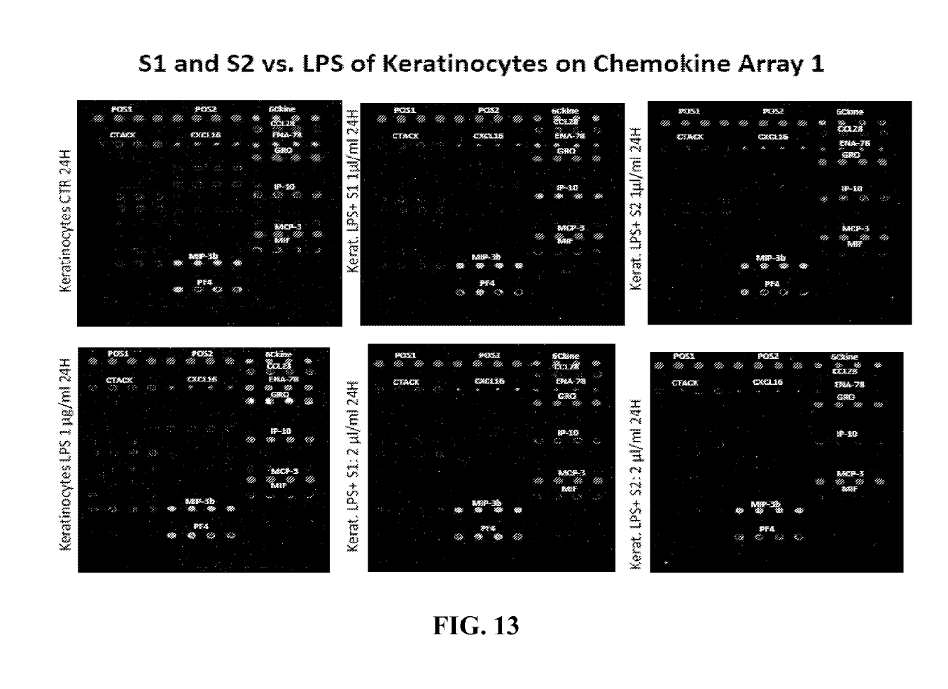

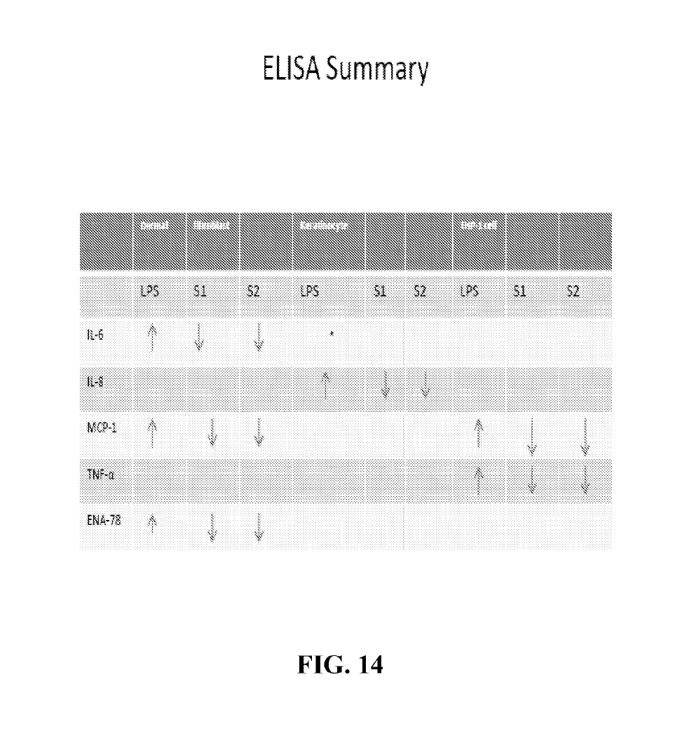

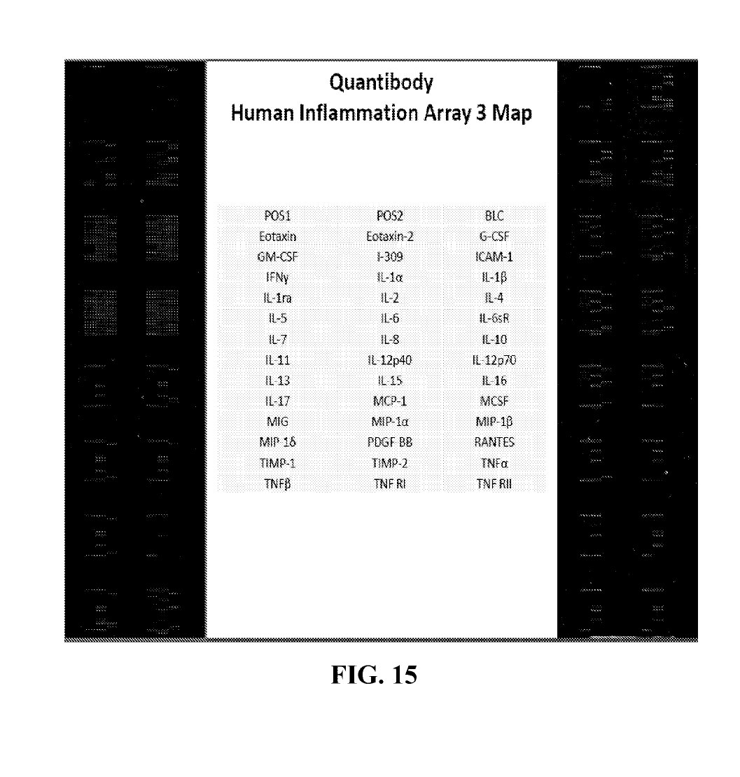

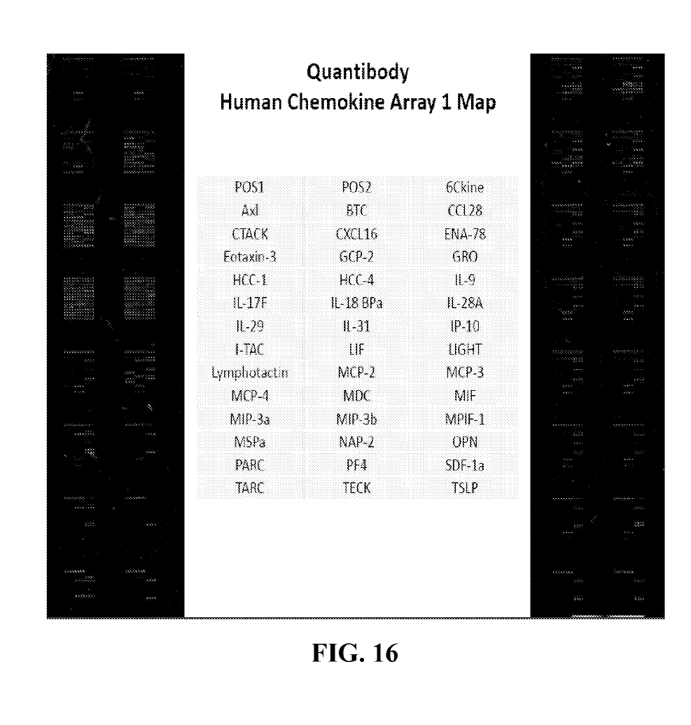

As shown in FIGS. 5-13, sandalwood oil has anti-inflammatory properties as shown by the inhibition of cytokine/chemokine expression after LPS challenge. Sandalwood also inhibits basal level production of chemokines/cytokines by dermal fibroblasts and keratinocytes.

### Treatment of Acne

A topical blend of salicylic acid (SA) and highly purified Australian sandalwood oil was used in an open-label study in adolescents and adults with mild to moderate facial acne.

The investigational regimen consisted of a foaming cleanser (Formulation D), acne Serum (Formulation C), a spot treatment (Formulation B), and a mask (Formulation A). Patients applied the treatment regimen as directed for 8 weeks. The primary efficacy measure was the percentage of patients assessed as Improved, Much Improved, or Very Much Improved according to the Global Aesthetic Improvement Scale (GAIS) ratings at week 8. Severity was rated using the Evaluator's Global Severity Scores (EGSS) at baseline and weeks 2, 4, and 8. Tolerability was assessed at baseline and weeks 2, 4, and 8 by asking patients to rate the severity of itching, scaling, erythema, burning, dryness, and stinging. Patients were also asked to complete a Daily Acne Questionnaire.

This study systematically examined the efficacy, safety, tolerability and compliance of a treatment regimen combining SA with sandalwood oil in adolescents and adults with facial acne.

Forty-seven patients completed 8 weeks of treatment. Forty-two patients (89%) met the primary endpoint determined by the GAIS of Improved (66%), Much Improved (19%), or Very Much Improved (4%). Notable reductions in lesion counts were observed in patients with more severe or inflamed lesions. Tolerability was queried at all visits. No itching, scaling, or erythema was reported after initial application. Symptoms of intolerability peaked at week 2; however, most events were mild to moderate and were typically reported with use of the Mask component. Intolerance decreased by week 4 and by week 8, the treatment regimen was well tolerated by patients.

### Methods

The Western Institutional Review Board (WIRB) approved the protocol and informed consent/assent documents before study start-up. All patients and legally authorized representatives (for minors) provided written informed consent/assent prior to conduct of any study-related activities.

### Patients

Patients ≧14 to ≦45 years of age with mild-to-moderate (≧8 inflammatory lesions) facial acne were enrolled. Exclusion criteria included use of any topical acne treatment (eg, benzoyl peroxide, salicylates, etc) in the weeks just prior to baseline; use of the following medications (time prior to baseline): systemic antibiotics (1 month); topical corticosteroids on affected areas (1 month); oral retinoids (3 months); or estrogens, topical contraceptives, or androgenic agents (3 months). Stable doses of estrogens, estrogen-containing products, androgens, or anti-androgenic products just prior to enrollment was permitted provided the dose remained the same throughout the study period. Patients undergoing excessive sun/ultraviolet light exposure during the study were also excluded.

### Study Design

This open-label study was conducted between June 2011 and April 2012 at a single center in the United States.

Study Drug Therapy. The investigational regimen consisted of: Foaming Cleanser (0.5% salicylic acid); Serum (0.5% salicylic acid); Spot Treatment (2% salicylic acid); and a Mask (0.5% salicylic acid). Products were applied to all blemishes in the treatment area, including any new blemishes according to the following schedule for 56 days: Foaming Cleanser—face and neck washed morning and night; Serum—face and neck treated each evening after cleansing; Spot Treatment—blemishes treated up to 3 to 4 times/day, as needed; and Mask—face and neck covered 3 times/ week, allowed to dry for at least 5 minutes, then removed with a clean cloth and warm water. When possible, the investigator applied the treatment regimen to the patient at the baseline visit to ensure correct use of the products. Compliance was assessed through review of the daily/weekly Acne questionnaire and patient query regarding the frequency of treatment applications. Patients were also instructed to bring all product containers to each study visit for weighing.

Efficacy Evaluations. The investigator assessed efficacy via lesion counts, Global Aesthetic Improvement Scale (GAIS) ratings (Narins et al. “A randomized, doubleblind, multicenter comparison of the efficacy and tolerability of Restylane versus Zyplast for the correction of nasolabial folds”, Dermatol Surg. 2003; 29:588-595) and Evaluator's Global Severity Scores (EGSS). Skin condition was rated at Weeks 2, 4, and 8 using GAIS categories: Very Much Improved; Much Improved; Improved; No Change; or Worse when compared to Baseline. Severity of acne was rated at baseline, weeks 2, 4, and 8 using EGSS (Table 3).

TABLE 3
Evaluator's Global Severity Score

| Score | Grade | Description |
|---|---|---|
| 0 | Clear | Normal, clear skin with no evidence of acne vulgaris |
| 1 | Almost | Rare noninflammatory lesions present, with rare |
| | clear | noninflamed papules (papules must be resolving and |
| | | may be hyperpigmented but not pink-red) |
| 2 | Mild | Some noninflammatory lesions present, with few |
| | | inflammatory lesions (papules/pustules only; no |
| | | nodulocystic lesions) |
| 3 | Moderate | Noninflammatory lesions predominating, with multiple |
| | | inflammatory lesions evident and several to many |
| | | comedones and papules/pustules; there may be 1 small |
| | | nodulocystic lesion |
| 4 | Severe | Inflammatory lesions more apparent, with many |
| | | comedones and papules/pustules; there may be a few |
| | | nodulocystic lesions |
| 5 | Very | Highly inflammatory lesions predominating, with |
| | severe | variable numbers of comedones, many papules/ |
| | | pustules, and many nodulocystic lesions |

Patients completed a Daily Acne Questionnaire (DAQ) starting after the first application of the investigational regimen and continuing for the first 7 days of treatment and weekly thereafter. Patients rated aspects of their treatment: 0=no improvement; 1=Slight Improvement; 2=Some Improvement; 3=Much Improved; and 4=Very Much Improved). Photographic evidence of disease severity and response was collected at each visit.

Safety Assessments. Adverse events (AEs) were monitored from the first application through Week 8 (or discontinuation visit), and 4 weeks after completion of the study for events considered serious and related to the investigational regimen. Cutaneous tolerability was also assessed at each visit, with patients asked to rate the severity of itching, scaling, erythema, burning, dryness, and stinging using a 4-point scale (0=none; 1=mild; 2=moderate; and 3=severe).

### Statistical Analysis

Efficacy Endpoints. The primary efficacy measure was the percentage of patients that exhibited a positive response to the investigational regimen according to the GAIS (ie, patients assessed as Very Much Improved, Much Improved, or Improved) at week 8. Secondary analyses included the percent change from baseline at week 8 in inflammatory lesions (ILs), non-inflammatory lesions (NILs), and total lesion counts, as well as the percentage of patients with a baseline EGSS of ‘Moderate’ or worse who: (1) were clear or almost clear (indicative of treatment success) at week 8; (2) had achieved an improvement of at least two grades in EGSS at week 8, and; (3) exhibited EGSS treatment success or achieved an improvement of at least two grades in EGSS at week 8.

Sample Size Determination and Data Analysis. Up to 50 patients were planned for enrollment to ensure a final sample size of 45 patients. Efficacy was assessed in those patients who completed 8 weeks of treatment (Per Protocol (PP) population). Patients who received at least one administration of the investigative regimen comprised the intent-to-treat (ITT) population used to assess safety and tolerability. Summary statistics were determined for baseline characteristics and continuous endpoints. Number and percent were calculated for categorical endpoints.

### Results

Patient Disposition and Completion. Fifty patients were enrolled from June 2011 to February 2012; 47 patients completed 8 weeks of treatment, thereby comprising the PP population. Three patients failed to return for study visits; two after Visit 1, and one Visit 2. No AEs, treatment discontinuations, or delays were noted in these three patients. Thus, the PP population matched the ITT population.

### Demographic and Other Baseline Characteristics

Most patients were non-Hispanic Caucasian females older than 18 years of age with mild-to-moderate acne indicated by an EGSS of 2.4. Demographics and baseline characteristics for the ITT population are presented in Table 4.

TABLE 4
Patient Demographics and Baseline Characteristics

| Patients | Count | Mean (Median) | Range |
|---|---|---|---|
| Age | | | |
| All | 47 | 23.3 (23) | 14-42 |
| Under 18 | 11 | 23.4 | 14-18 |
| Sex | | | |
| Male | 14 (30%) | | |
| Female | 33 (70%) | | |
| Race | | | |
| Asian | 11 (23%) | | |
| African American | 2 (4%) | | |
| Caucasian | 33 (70%) | | |
| Nonhispanic | 33 (70%) | | |
| Other | 1 (2%) | | |
| Lesion count at baseline | | | |
| Non-inflammatory | | 23.6 (17) | 6-94 |
| Inflammatory | | 20.1 (16) | 8-66 |
| Total | | 43.5 (34) | 18-118 |
| EGSS* | | 2.4 (2) | 1-4 |

*Evaluator Global Severity Score

Compliance and Concomitant Anti-acne Medication Usage. Every effort was made to include all enrolled patients in the PP population. All patients who returned the treatment products at their study visits with demonstrated usage were considered compliant. Patients who failed to return their treatment products had the last weights of the product containers carried forward to apply the most conservative estimate of usage after 8 weeks of treatment. Use of the cleanser (twice/day) was considered the best indicator of product usage since use of the other products was expected to vary more during the study. None of the completed patients reported using any acne treatment other than the investigational regimen provided by the study.

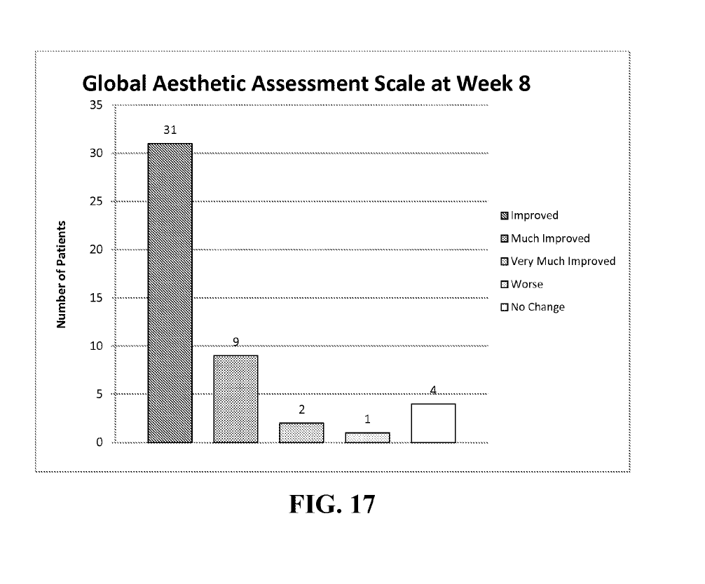

Clinical Response. Forty-two (89.4%) of the 47 patients who completed 8 weeks of treatment met the primary endpoint determined by the GAIS of Improved (31 of 47; 66.0%), Much Improved (9 of 47; 19.1%), or Very Much Improved (2 of 47; 4.3%) (FIG. 17). One patient (2.1%) whose acne was worse at week 8 was noted to have ‘Improved’ at the week 4 visit, but used a new cosmetic powder that caused irritation and a local skin reaction that was present at the week 8 assessment. Four patients exhibited no change.

Of the four patients (8.5%) who had no change in GAIS at week 8, two had decreased lesion counts at week 8, but had experienced greater decreases during earlier visits. Two other patients had no/minimal changes in lesion counts, one of whom experienced a reduction in lesion counts and overall improvement in her acne, but not substantial enough to change the GAIS.

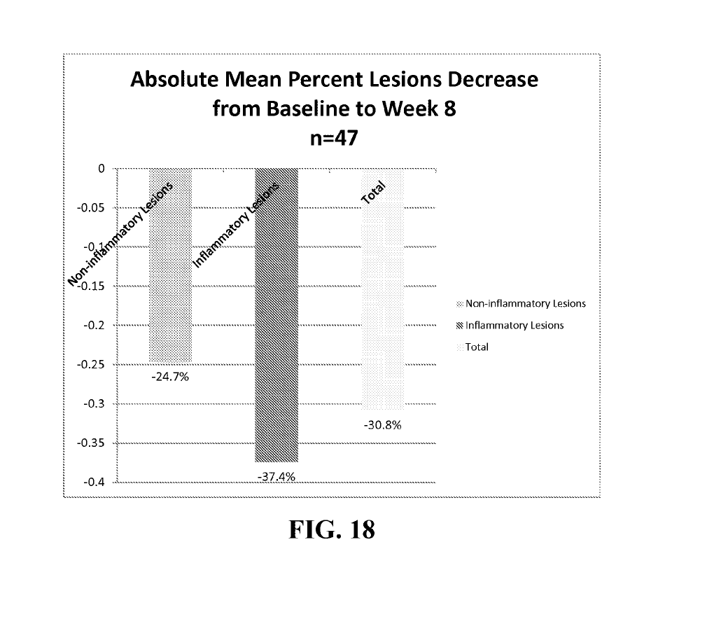

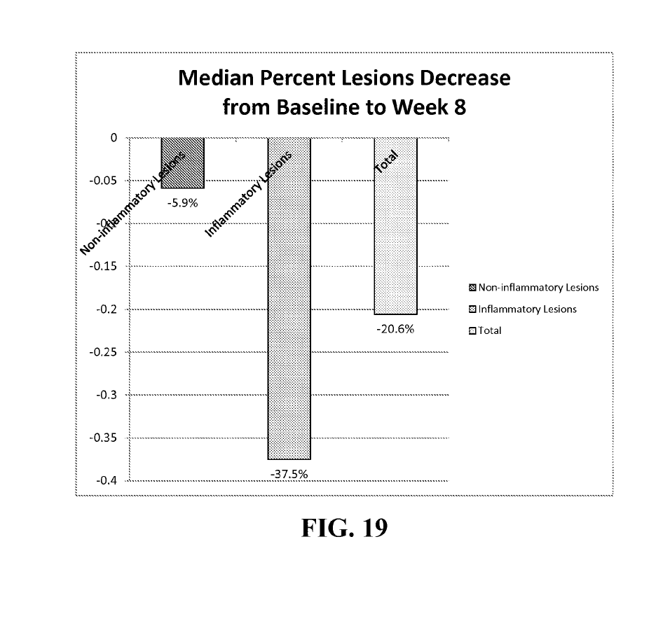

The investigator rated the patient's acne using EGSS. At Week 8, 27 patients (57.5%) had no change in EGSS; 18 (38.3%) improved by at least one score; 2 (4.2%) improved by two scores and 2 patients (4.2%) worsened. One patient (2.1%) improved from 66 ILs at baseline to 3 ILs at week 8. Mean and median changes in lesion counts from baseline to week 8 are shown for patients in the PP population (FIG. 18, FIG. 19, respectively).

Notable reductions in lesion counts were observed in patients with more severe or inflamed lesions. A 31% reduction in ILs was observed (mean of 20 ILs at baseline to 13 ILs at week 8).

### Safety and Tolerability

Adverse events (AEs) were reported by the patients and solicited by the investigator at each study visit. Three AEs were reported by three separate patients (6.4%) including a moderate local skin reaction considered unlikely related; a mild reaction to an influenza vaccine considered unrelated; and an instance of moderate dizziness considered unlikely related to the investigational regimen. All AEs resolved and none was considered serious.

Tolerability was queried at all visits including after the initial application of the investigational regimen (Table 5). No itching, scaling, or erythema was reported after initial application of the investigational regimen. The number of patients reporting symptoms of intolerability peaked at week 2; however, most events were mild to moderate in severity and were typically reported with use of the Mask component. Intolerance decreased by week 4 and by week 8, the treatment regimen was well tolerated by nearly all of patients.

TABLE 5
Patient Reported Tolerability Evaluations

| | Baseline* | Week 2 | Week 4 | Week 8* |
|---|---|---|---|---|
| Symptom | (n = 46) | (n = 47) | (n = 47) | (n = 46) |
| Itching | | | | |
| 0 | 46 (100%) | 47 (100%) | 46 (98%) | 45 (98%) |
| 1 | — | — | 1 (2%) | 1 (2%) |
| 2 | — | — | — | — |
| 3 | — | — | — | — |
| Scaling | | | | |
| 0 | 46 (100%) | 43 (92%) | 45 (96%) | 46 (100%) |
| 1 | — | 2 (4%) | 2 (4%) | — |
| 2 | — | 1 (2%) | — | — |
| 3 | — | — | — | — |
| Erythema | | | | |
| 0 | 46 (100%) | 43 (92%) | 44 (94%) | 44 (96%) |
| 1 | — | 2 (4%) | 2 (4%) | — |
| 2 | — | — | 1 (2%) | 1 (2%) |
| 3 | — | 1 (2%) | — | 1 (2%) |
| Burning | | | | |
| 0 | 32 (70%) | 29 (62%) | 35 (74%) | 41 (89%) |
| 1 | 8 (17%) | 15 (32%) | 9 (19%) | 3 (7%) |
| 2 | 5 (11%) | 3 (6%) | 3 (6%) | 2 (4%) |
| 3 | 1 (2%) | — | — | — |
| Dryness | | | | |
| 0 | 43 (94%) | 39 (83%) | 41 (87%) | 42 (91%) |
| 1 | 2 (4%) | 5 (11%) | 3 (6%) | 1 (2%) |
| 2 | 1 (2%) | 3 (6%) | 2 (4%) | 2 (4%) |
| 3 | — | — | 1 (2%) | — |
| Stinging | | | | |
| 0 | 38 (83%) | 39 (83%) | 41 (87%) | 45 (98%) |
| 1 | — | 5 (11%) | 5 (11%) | — |
| 2 | — | 3 (6%) | — | 1 (2%) |
| 3 | 8 (17%) | — | 1 (2%) | — |

*Only 46 of 47 patients completed the tolerability evaluations at the Baseline and Week 8 visits.
0 = none; 1 = mild; 2 = moderate; 3 = severe

Approximately 86% of patients completed and turned in their patient diaries. Responses are summarized by time point in Table 6.

TABLE 6
Percentage of Patients Self-reporting Improvement in Patient Diaries

| Diary Questions | Day 1 | Day 2 | Day 3 | Day 4 | Day 5 | Day 7 | Week 2 | Week 4 | Week 8 |
|---|---|---|---|---|---|---|---|---|---|
| Do you see | 15% | 40% | 75% | 71% | 78% | 85% | 93% | 81% | 90% |
| Improvement in your | | | | | | | | | |
| complexion since | | | | | | | | | |
| starting treatment? | | | | | | | | | |
| Do you feel any | 35% | 70% | 85% | 78% | 73% | 88% | 93% | 83% | 93% |
| improvement in your | | | | | | | | | |
| complexion since | | | | | | | | | |
| starting treatment? | | | | | | | | | |
| Do you feel the | 20% | 48% | 68% | 63% | 78% | 78% | 85% | 86% | 93% |
| treatment regimen is | | | | | | | | | |
| making a significant | | | | | | | | | |
| improvement in your | | | | | | | | | |
| acne? | | | | | | | | | |
| Does your face feel | 78% | 93% | 93% | 88% | 85% | 95% | 95% | 97% | 100% |
| cleaner since starting | | | | | | | | | |
| the treatment regimen? | | | | | | | | | |
| If your face less | 35% | 63% | 63% | 63% | 68% | 66% | 78% | 86% | 85% |
| painful/irritated since | | | | | | | | | |
| starting the treatment | | | | | | | | | |
| regimen? | | | | | | | | | |
| Do you feel better and | 33% | 53% | 63% | 89% | 53% | 61% | 78% | 81% | 90% |
| more confident since | | | | | | | | | |
| you have started the | | | | | | | | | |
| treatment regimen? | | | | | | | | | |
| Have your friends or | 10% | 15% | 28% | 22% | 39% | 46% | 66% | 75% | 90% |
| relatives commented on | | | | | | | | | |
| improvements in your | | | | | | | | | |
| complexion? | | | | | | | | | |

*Patient responses to the questions included one of the following: Slight Improvement; Some Improvement; Much Improved; or Very Much Improved.

After initial application of the investigational regimen, more than 75% of patients reported that their faces felt cleaner and approximately 33% of patients seeing improvements in their complexion, less pain/irritation, and more confidence since starting the acne treatment.

After the first week, more than 75% of patients reported seeing improvements in their complexion, feeling that the investigational regimen is significantly improving their acne, and that their faces felt cleaner. Sixty-one percent of patients also reported less pain/irritation since starting the regimen. By week 4, more than 80% of patients reported seeing and feeling improvements in their complexion, that the treatment was significantly improving their acne; that their skin felt cleaner and less irritated; and that they had more confidence since starting the investigational treatment regimen.

At the end of the study (week 8), over 85% of patients reported improvements in all diary categories.

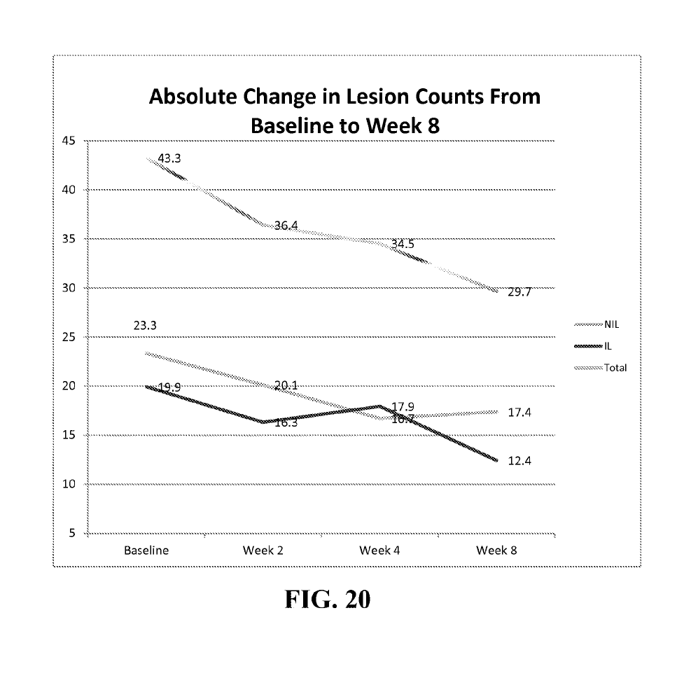

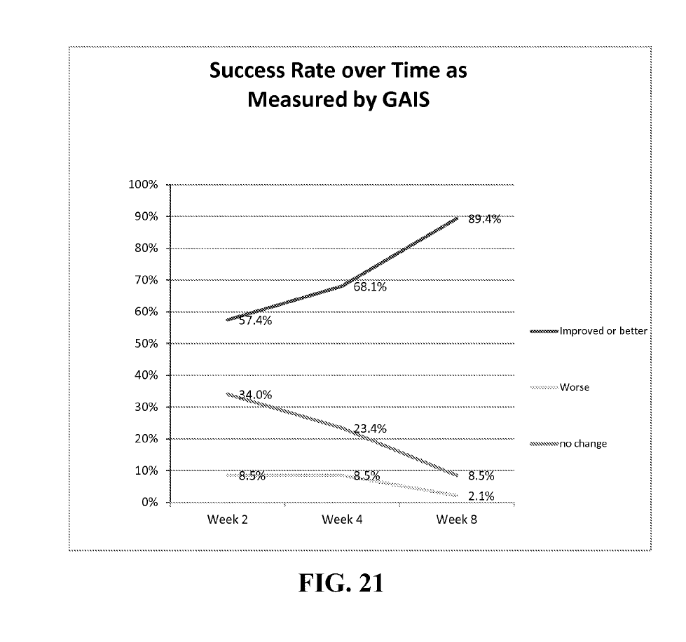

This single center, open-label study with a topical SA-based investigational regimen containing highly purified Australian sandalwood oil was conducted in adolescents and adults with mild-to-moderate facial acne. Approximately 89% of patients showed improvements in their disease (ie, Improved, Much Improved, and Very Much Improved) when compared to baseline. Absolute mean decreases were observed in the numbers of ILs (37%), NILs (25%), and total lesion counts (31%) (FIG. 18) with impressive self-reported results from the patient diaries (improvement in the sight, cleanliness and feel of skin and improved self confidence observed in ≧85% of patients across all categories). Of notable importance was the continued improvement seen throughout the 8-week treatment period, which strongly suggests that continued use of this treatment regimen could result in continued improvements and increased efficacy (FIGS. 20 and 21).

No adverse effects were observed that would limit use or constitute significant intolerability. Although some intolerance was reported during this study, it was generally mild. Interestingly, the tolerability issues seen in this study differed from those typically observed with other OTC acne products in that they did not intensify with continued usage.

This study demonstrates that the formulations set forth herein are efficacious. Further, these formulations have a favorable adverse event and tolerability profile that could be readily accepted by patients with acne and provide clinicians with an alternative treatment regimen for the treatment of acne.

1. A composition comprising:
(a) a therapeutically effective amount of sandalwood heartwood oil;
(b) sandalwood nut oil.

2. The composition of claim 1, further comprising an active agent selected from the group consisting of acetic acid, acetone, alcloxa, alcohol, alkyl isoquinolinium bromide, allantoin, allyl isothiocyanate, aloe vera, alum, aluminum chlorohydrex, aluminum hydroxide, aluminum sulfate, amiloxate, ammonia solution, ammoniated mercury, amyltricresois, an antibiotic, ascorbic acid, aspirin, bacitracin, basic fuchsin, beeswax, benzalkonium chloride, benzethonium chloride, benzocaine, benzoic acid, benzoxiquine, benzoyl peroxide, benzyl alcohol, bismuth subsalicylate, boric acid, calcium polysulfide, calcium thiosulfate, calcium undecylate, calcium undecylenate, calomel, camphor, camphorated metacresol, candicin, captan, chloral hydrate, chlorhydroxyquinoline (cloxyquin), chlorobutanol, chlorothymol, chloroxylenol, clioquinol, cloflucarban, clotrimazole, coal tar, colloidal oatmeal, copper undecylate, cresol, cyclomethycaine sulfate, dibenzothiophene, dichlorophen,erythromycin, estrone, ethohexadiol, eucalyptol, eugenol, fluorosalan, glycerin, haloprogin, hexachlorophene, hexylresorcinol, hydrocortisone preparations, hydrogen peroxide, iodine-containing ingredient, juniper tar, kaolin, lanolin, lauryl isoquinolinium bromide, mandelic acid, magnesium aluminum silicate, magnesium sulfate, m-cresol, methylparaben, menthol, merbromin, mercufenol chloride, mercuric chloride, mercuric oxide, mercuric salicylate, mercuric sulfide (red), mercury, mercury oleate, mercury sulfide, mercury-containing ingredient, methapyrilene hydrochloride, methyl salicylate, methylbenzethonium chloride, miconazole nitrate, mineral oil, neomycin, nitromersol, nonylphenoxypoly ethanol iodine, nystatin, oxyquinoline, oxyquinoline, oxyquinoline sulfate, PABA, para-chloromercuriphenol, peppermint oil, phenol, phenolate sodium, phenyl salicylate, phenylmercuric nitrate, pine tar, poloxamer 188, poloxamer-iodine complex, potassium, povidone-iodine, propionic acid, propylparaben, pyrilamine maleate, pyrithione zinc, resorcinol, resorcinol monoacetate, salicylamide, salicylic acid, selenium sulfide, shark liver oil, sodium borate, sodium caprylate, sodium propionate, sodium salicylate, sodium thiosulfate, sulfur, tannic acid, tetracaine, tetracaine hydrochloride, tetracycline, thimerosal, thymol, tolindate, tolnaftate, triacetin, triclocarban, triclosan, triple dye, undecoylium chlorideiodine complex, undecylenic acid, vitamin A, vitamin E, vitromersal, zinc acetate, zinc caprylate, zinc oxide, zinc propionate, zinc stearate, zinc sulfide, zinc undecylate, zyloxin and 2-ethylhexyl-4-phenylbenzopohenone-2-carboxylic acid.

3. (canceled)

4. (canceled)

5. The composition of claim 1, wherein the concentration of the sandalwood heartwood oil is greater than about 0.3% (w/w) to about 10% (w/w).

6. The composition of claim 1, wherein the concentration of the sandalwood nut oil is from about 0.5% to about 10% (w/w).

7. The composition of claim 2, wherein the concentration of the active ingredient is from about 0.25% to about 25% (w/w).

8. The composition of claim 1, further comprising a fruit acid.

9. (canceled)

10. The composition of claim 1, further comprising a fruit extract.

11. (canceled)

12. The composition of claim 1, further comprising a flower extract.

13. (canceled)

14. The composition of claim 1, further comprising a solvent.

15. (canceled)

16. The composition of claim 14, wherein the solvent is water or alcohol.

17. (canceled)

18. (canceled)

19. (canceled)

20. (canceled)

21. (canceled)

22. (canceled)

23. The composition of claim 1, wherein the composition is in a form selected from the group consisting of an ointment, lotion, cream, moisturizer, gel, mousse, clay mask, serum, cleanser, shaving cream, shaving gel, soap, shampoo and a stick.

24. (canceled)

25. (canceled)

26. A method of treating a skin disorder in a subject comprising administering the composition of claim 1 to the subject, where the subject has a skin disorder or is at risk of developing a skin disorder.

27. The method of claim 26, wherein the skin disorder is selected from the group consisting of acne, rosacea, psoriasis, eczema, dermatitis, dandruff, diaper rash and fungal infection.

28. (canceled)

29. (canceled)

30. (canceled)

31. An anti-acne composition comprising:
(a) an active anti-acne agent;
(b) a fruit acid;
(c) sandalwood heartwood oil;
(d) a fruit extract; and
(e) a flower extract.

32. The composition of claim 31, wherein the active anti-acne agent is selected from the group consisting of salicylic acid, benzoyl peroxide, glycolic acid and sulfur.

33. (canceled)

34. (canceled)

35. The composition of claim 31, wherein the concentration of the heartwood oil is from about 0.25% to about 10% (w/w).

36. (canceled)

37. (canceled)

38. The composition of claim 31, further comprising a solvent.

39. (canceled)

40. (canceled)

41. (canceled)

42. (canceled)

43. (canceled)

44. (canceled)

45. (canceled)

46. (canceled)

47. (canceled)

48. An anti-acne composition comprising:
(a) an active anti-acne agent;
(b) sandalwood heartwood oil;
(c) flower extract; and
(d) lemon myrtle leaf extract.

49. The composition of claim 48, wherein the anti-acne agent is selected from the group consisting of salicylic acid, benzoyl peroxide, glycolic acid and sulfur.

50. (canceled)

51. (canceled)

52. (canceled)

53. (canceled)

54. The composition of claim 48, further comprising a solvent.

55. (canceled)

56. (canceled)

57. (canceled)

58. The composition of claim 31, wherein the composition is in a form selected from the group consisting of an ointment, lotion, cream, moisturizer, gel, mousse, clay mask, serum, cleanser, shaving cream, shaving gel, shampoo, soap and a stick.

59. (canceled)

60. A method of treating acne in a subject comprising administering the composition of claim 31 to the subject, wherein the subject has acne or is at risk of developing acne.

61. (canceled)

62. (canceled)
